(19) United States
(12) Patent Application Publication
(10) Pub. No.: US 2024/0285818 A1
(43) Pub. Date: Aug. 29, 2024

(54) RADIOIMMUNOCONJUGATES AND CHECKPOINT INHIBITOR COMBINATION THERAPY

(71) Applicant: Fusion Pharmaceuticals Inc., Hamilton (CA)

(72) Inventors: Eric Steven Burak, Cambridge (CA); Julie Metcalf, Brantford (CA); John Fitzmaurice Valliant, Ancaster (CA); Meiduo Hu, Maple (CA); Natalie Grinshtein, Bothell, WA (US)

(21) Appl. No.: 18/568,234

(22) PCT Filed: Jun. 10, 2022
(86) PCT No.: PCT/US22/33058

§ 371 Date: Dec. 7, 2023

Related U.S. Application Data
(60) Provisional application No. 63/209,736, filed on Jun. 11, 2021.

(51) Int. Cl. A61K 51/10; A61K 39/395; A61P 35/00

(52) U.S. Cl. CPC A61K 51/1096; A61K 39/3955; A61K 51/1027; A61K 51/103; A61P 35/00; A61K 2121/00

(57) ABSTRACT

Combination therapies comprising administering radioimmunoconjugates and one or more checkpoint inhibitors.

### RELATED APPLICATIONS

The present application claims priority to U.S. Provisional Patent Application No. 63/209,736, filed Jun. 11, 2021, the entire contents of which are hereby incorporated by reference for all purposes.

### SEQUENCE LISTING

The present specification makes reference to a Sequence Listing (submitted electronically as a .txt file named “FPI_021_Sequence_Listing.txt” on Jun. 10, 2022). The .txt file was generated on Jun. 9, 2022 and is 19.2 kilobytes in size. The entire contents of the Sequence Listing are herein incorporated by reference.

### BACKGROUND

Cancer cells employ a variety of mechanisms to escape immune surveillance, including suppression of T cell activation.

The mammalian immune system relies on checkpoint molecules to distinguish normal cells from foreign cells. Checkpoint molecules, expressed on certain immune cells, need to be activated or inactivated to start an immune response. Inhibition of checkpoint proteins results in increased activation of the immune system.

Checkpoint inhibition has been explored as a method of immunotherapy for cancer. Inhibiting checkpoint proteins may activate T-cells and allow them to attack cancer cells. However, checkpoint inhibition can allow the immune system to attack some normal cells in the body, which can lead to serious side effects. In addition, some checkpoint inhibitors have exhibited only modest efficacy in the clinic. There remains a need for improved treatments of cancer. In particular, there is a need for increases in efficacy, which do not enhance toxicity in the patient.

### SUMMARY

The present disclosure encompasses the insight that combining inhibition of checkpoint proteins with a therapy that targets damage to cancer cells may provide a less toxic therapy with improved efficacy. Radioactive decay can cause direct physical damage (such as single or double-stranded DNA breaks) or indirect damage (such as by-stander or crossfire effects) to the biomolecules that constitute a cell. The present disclosure combines radioimmunoconjugates targeted to cancer cells with checkpoint inhibition to induce or improve an immune response to a tumor. In some embodiments, disclosed combination therapies ameliorate or treat cancer.

In one aspect, provided are methods of treating a patient having cancer, said method comprising administering to the patient a therapeutically effective amount of an [²²⁵Ac]-radioimmunoconjugate, which comprises ²²⁵Ac chelated with a compound having the formula: A-L¹-X-L²-Z—B,
wherein A is a chelating moiety selected from DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (hydroxypropyltetraazacyclododecanetriacetic acid);
L¹ is optionally substituted C₁₋₆ alkyl or C₁₋₆ heteroalkyl;
X is C═O(NR¹), NR¹C═O(O), NR¹C═O(NR¹), CH₂PhC═O(NR¹), O, or NR¹, each R¹ independently being H or C₁₋₆ alkyl;
L² is optionally substituted C₁₋₅₀ alkyl or C₁₋₅₀ heteroalkyl;
Z is C═O, CH₂, OC═O, NR²C═O, or NR², each R² independently being H or C₁₋₆ alkyl; and
B is a targeting moiety,
wherein the patient has received or is receiving one or more checkpoint inhibitors, and wherein the [²²⁵Ac]-radioimmunoconjugate is administered at a dose of about 10 kBq to about 400 kBq/kg of body weight of said patient or is administered as a unitary dosage of about 1-30 MBq to said patient.

In some embodiments, the above compound has the chelating moiety being DOTA.

In some embodiments, the compound has formula I:

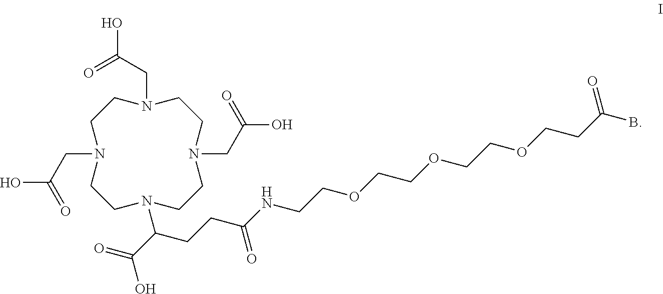

In some embodiments, the compound has formula II:

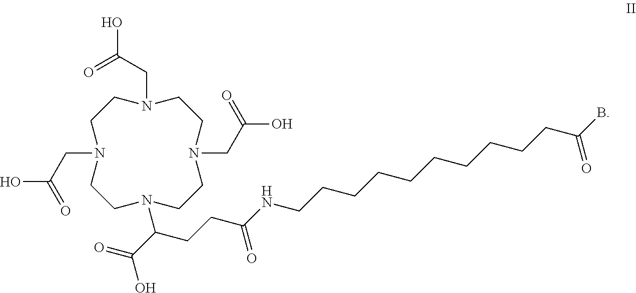

In some embodiments, the targeting moiety comprises an antibody or antigen-binding fragment thereof.

In some embodiments, B is an insulin-like growth factor 1 receptor (IGF-1R) antibody or antigen-binding fragment thereof, an endosialin (TEM-1) antibody or antigen-binding fragment thereof, or a fibroblast growth factor receptor 3 (FGFR3) antibody or antigen-binding fragment thereof.

In some embodiments, B is an IGF-1R antibody or antigen-binding fragment thereof selected from the group consisting of figitumumab, cixutumumab, TAB-199, AVE1642, BIIB002, robatumumab, and teprotumumab, and antigen-binding fragments thereof.

In some embodiments, B is AVE1642 or an antigen-binding fragment thereof.

In some embodiments, the [²²⁵Ac]-radioimmunoconjugate is administered at a dose of about 10 kBq to about 200 kBq/kg (e.g., about 10 kBq to about 150 kBq/kg, about 10 kBq to about 120 kBq/kg, about 10 kBq to about 100 kBq/kg, about 30 kBq to about 150 kBq/kg, about 30 kBq to about 120 kBq/kg, about 30 kBq to about 100 kBq/kg, about 40 kBq to about 120 kBq/kg, about 40 kBq to about 100 kBq/kg, or about 40 kBq to about 80 kBq/kg) of body weight of said patient.

In some embodiments, the [²²⁵Ac]-radioimmunoconjugate is administered as a unitary dosage of about 1-30 MBq (e.g., about 2-25 MBq, about 3-20 MBq, about 5-15 MBq, about 8-12 MBq, or about 10 MBq) to said patient.

In some embodiments, the one or more checkpoint inhibitors comprise a PD-1 inhibitor, a CTLA-4 inhibitor, or a combination thereof.

In some embodiments, the one or more checkpoint inhibitors comprise both a PD-1 inhibitor and a CTLA-4 inhibitor.

In some embodiments, the PD-1 inhibitor or the CTLA-4 inhibitor is an antibody.

In some embodiments, the one or more checkpoint inhibitors comprise a PD-1 inhibitor administered at a dose of about 5 mg/kg to about 15 mg/kg.

In some embodiments, the PD-1 inhibitor is pembrolizumab.

In some embodiments, the one or more checkpoint inhibitors comprise both a PD-1 inhibitor and a CTLA-4 inhibitor, each administered at a dose of about 5 mg/kg to about 15 mg/kg (e.g., about 6 mg/kg, about 7 mg/kg, about 8 mg/kg, about 9 mg/kg, about 10 mg/kg, about 11 mg/kg, about 12 mg/kg, about 13 mg/kg, or about 14 mg/kg).

In some embodiments, B is AVE1642 or an antigen-binding fragment thereof, and the one or more checkpoint inhibitors comprise a PD-1 inhibitor that is pembrolizumab.

In some embodiments, the [²²⁵Ac]-radioimmunoconjugate is administered at a dose of about 30 kBq to about 120 kBq/kg of body weight of said patient, and the PD-1 inhibitor administered at a dose of about 5 mg/kg to about 15 mg/kg.

In some embodiments, the patient has a cancer selected from the group consisting of breast cancer (e.g., triple negative breast cancer or TNBC), non-small cell lung cancer, small cell lung cancer, pancreatic cancer, head and neck cancer, prostate cancer, colorectal cancer, cervical cancer, endometrial cancer, sarcoma, adrenocortical carcinoma, neuroendocrine cancer, Ewing's Sarcoma, multiple myeloma, and acute myeloid leukemia.

In some embodiments, the patient has a solid tumor expressing IGF-1R.

In some embodiments, B is capable of binding to a tumor-associated antigen and said administering results in an increase in CD8+ T cells specific for the tumor-associated antigen.

In some embodiments, said administering results in at least 60% of the total CD8+ T cell population in a sample from the patient being specific for the tumor-associated antigen. In some embodiments, the sample is a tumor sample.

### BRIEF DESCRIPTION OF THE DRAWINGS

FIG. 1 illustrates relative tumor volume in the CT26 syngeneic mouse tumor model after treatment with various checkpoint inhibitors. Relative tumor volume at various timepoints after treatment initiation is shown for vehicle control, anti-PD-1 isotype control (15 mg/kg), anti-PD-1 (5 mg/kg or 15 mg/kg), anti-CTLA-4 isotype control (15 mg/kg), and anti-CTLA-4 (5 mg/kg or 15 mg/kg) treatment groups.

FIG. 2 illustrates [¹⁷⁷Lu]-Compound B biodistribution in the CT-26 syngeneic mouse tumor model. Shown are the percentage of injected dose (% ID) per gram in blood, bone, intestines, kidneys and adrenals, liver and gall bladder, lungs, spleen, tumor, and urine and bladder at 4 hours, 24 hours, 48 hours, 96 hours, and 168 hours.

FIG. 3 illustrates the enhanced efficacy of [²²⁵Ac]-Compound C in immunocompetent mice vs. immunodeficient mice. Relative tumor volumes at various timepoints after treatment initiation are shown for control and treatment groups (50 nCi or 400 nCi [²²⁵Ac]-Compound C).

FIG. 4A illustrates synergy between [²²⁵Ac]-Compound C and α-CTLA-4/PD-1 treatment in the CT26 syngeneic mouse model. Relative tumor volumes at various timepoints after treatment initiation are shown for control (buffer) and treatment groups (anti-CTLA-4 (5 mg/kg), anti-PD-1 (5 mg/kg), 200 nCi [²²⁵Ac]-Compound C, 200 nCi [²²⁵Ac]-Compound C with anti-CTLA-4, 200 nCi [²²⁵Ac]-Compound C with anti-PD-1, or 200 nCi [²²⁵Ac]-Compound C with anti-CTLA-4 and anti-PD-1).

FIG. 4B illustrates synergy between [²²⁵Ac]-Compound D and α-CTLA-4/PD-1 treatment in the CT26 syngeneic mouse model. Relative tumor volumes at various timepoints after treatment initiation are shown for control (vehicle or cold human IGF-1R antibody) and treatment groups (anti-CTLA-4 (5 mg/kg), anti-PD-1 (5 mg/kg), 200 nCi [²²⁵Ac]-Compound C, 200 nCi [²²⁵Ac]-Compound C with anti-CTLA-4, 200 nCi [²²⁵Ac]-Compound C with anti-PD-1, or 200 nCi [²²⁵Ac]-Compound C with anti-CTLA-4 and anti-PD-1).

FIG. 5 illustrates the development of protective immunity in [²²⁵Ac]-Compound C treated mice upon CT26 re-challenge. Relative tumor volumes at various timepoints after re-challenge are shown for control and treatment groups ([²²⁵Ac]-Compound C, [²²⁵Ac]-Compound C with anti-PD-1, [²²⁵Ac]-Compound C with anti-CTLA-4, or [²²⁵Ac]-Compound C with anti-CTLA-4 and anti-PD-1).

FIG. 6 illustrates a process for evaluating cytokine response and T-cell recruitment after [²²⁵Ac]-Compound C treatment.

FIG. 7 illustrates “humanized” IGF-1R model development. Shown are Western blots probed for expression of hIGF-1R in samples from CT26 cells that were stably transfected with human IGF-1R plasmid.

FIG. 8A is a schematic depicting the general structure of a bifunctional chelate comprising a chelate, a linker, and a cross-linking group. FIG. 8B is a schematic depicting the general structure of a bifunctional conjugate comprising a chelate, a linker, and a targeting moiety.

FIG. 9A illustrates synergy between [²²⁵Ac]-Compound D1 and α-CTLA-4/PD-1 treatment in the CT26 syngeneic mouse model. Relative tumor volumes at various timepoints after treatment initiation are shown for control (vehicle) and treatment groups (200 nCi [²²⁵Ac]-Compound D1, 200 nCi [²²⁵Ac]-Compound D1 with anti-PD-1, 200 nCi [²²⁵Ac]-Compound D1 with anti-CTLA-4, or 200 nCi [²²⁵Ac]-Compound C with anti-CTLA-4 and anti-PD-1).

FIG. 9B illustrates synergy between [²²⁵Ac]-Compound D2 and α-CTLA-4/PD-1 treatment in the CT26 syngeneic mouse model. Relative tumor volumes at various timepoints after treatment initiation are shown for control (vehicle) and treatment groups (200 nCi [²²⁵Ac]-Compound D2, 200 nCi [²²⁵Ac]-Compound D2 with anti-PD-1, 200 nCi [²²⁵Ac]-Compound D2 with anti-CTLA-4, or 200 nCi [²²⁵Ac]-Compound D2 with anti-CTLA-4 and anti-PD-1).

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The present disclosure relates to combination therapies for inducing or improving an immune response to cancer using [²²⁵Ac]-radioimmunoconjugates and checkpoint inhibitors. In some embodiments, use of methods disclosed herein results in treatment or amelioration of cancer.

In some embodiments, a lower effective dose of the [²²⁵Ac]-radioimmunoconjugates and/or of the checkpoint inhibitor is used.

Radiolabeled targeting moieties (also known as radioimmunoconjugates) are designed to target a protein or receptor that is upregulated in a disease state and/or specific to diseased cells (e.g., tumor cells) to deliver a radioactive payload to damage and kill cells of interest. “Radioimmunotherapy,” when used herein, refers to a method of using a radioimmunoconjugate, such as one described below, to produce a therapeutic effect. Radioactive decay of the payload produces an alpha, beta, or gamma particle or Auger electron that can cause direct effects to DNA (such as single or double stranded DNA breaks) or indirect effects such as by-stander or crossfire effects.

Radioimmunoconjugates typically contain a targeting moiety (e.g., an antibody or antigen binding fragment thereof, peptide, or small molecule that specifically binds to a molecule expressed on or by a tumor, e.g., IGF-1R, FGFR3, or TEM-1/endosialin), a chelating moiety or a metal complex of a chelating moiety (e.g., comprising a radioisotope), and a linker. Conjugates may be formed by appending a bifunctional chelate to a targeting molecule so that structural alterations are minimal while maintaining target affinity. A radioimmunoconjugate may be formed by radiolabeling such a conjugate.

Bifunctional chelates structurally contain a chelate, a linker, and a cross-linking group. Several examples of bifunctional chelates have been described with various cyclic and acyclic structures conjugated to a targeted moiety. [Bioconjugate Chem. 2000, 11, 510-519, Bioconjugate Chem. 2012, 23, 1029-1039, Mol Imaging Biol. 2011, 13, 215-221, Bioconjugate Chem. 2002, 13, 110-115].

### Definitions

### Chemical Terms

The term “acyl,” as used herein, represents a hydrogen or an alkyl group (e.g., a haloalkyl group), as defined herein, that is attached to the parent molecular group through a carbonyl group, as defined herein, and is exemplified by formyl (i.e., a carboxyaldehyde group), acetyl, trifluoroacetyl, propionyl, butanoyl and the like. Exemplary unsubstituted acyl groups include from 1 to 7, from 1 to 11, or from 1 to 21 carbons. In some embodiments, the alkyl group is further substituted with 1, 2, 3, or 4 substituents as described herein.

The term “alkyl,” as used herein, is inclusive of both straight chain and branched chain saturated groups from 1 to 20 carbons (e.g., from 1 to 10 or from 1 to 6), unless otherwise specified. Alkyl groups are exemplified by methyl, ethyl, n- and iso-propyl, n-, sec-, iso- and tert-butyl, neopentyl, and the like, and may be optionally substituted with one, two, three, or, in the case of alkyl groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxy, optionally substituted with an O-protecting group; (9) nitro; (10) oxo (e.g., carboxyaldehyde or acyl); (11) C₁₋₇ spirocyclyl; (12) thioalkoxy; (13) thiol; (14) —CO₂R^{A′}, optionally substituted with an O-protecting group and where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alk-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alk-C₆₋₁₀ aryl, and (d) hydroxy; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alk-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)_{S3}NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein. For example, the alkylene group of a C₁-alkaryl can be further substituted with an oxo group to afford the respective aryloyl substituent.

The term “alkylene” and the prefix “alk-,” as used herein, represent a saturated divalent hydrocarbon group derived from a straight or branched chain saturated hydrocarbon by the removal of two hydrogen atoms, and is exemplified by methylene, ethylene, isopropylene, and the like. The term “C_{x-y} alkylene” and the prefix “C_{x-y} alk-” represent alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁₋₆, C₁₋₁₀, C₂₋₂₀, C₂₋₆, C₂₋₁₀, or C₂₋₂₀ alkylene). In some embodiments, the alkylene can be further substituted with 1, 2, 3, or 4 substituent groups as defined herein for an alkyl group.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, 2-butenyl, and the like. Alkenyls include both cis and trans isomers. Alkenyl groups may be optionally substituted with 1, 2, 3, or 4 substituent groups that are selected, independently, from amino, aryl, cycloalkyl, or heterocyclyl (e.g., heteroaryl), as defined herein, or any of the exemplary alkyl substituent groups described herein.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, 1-propynyl, and the like. Alkynyl groups may be optionally substituted with 1, 2, 3, or 4 substituent groups that are selected, independently, from aryl, cycloalkyl, or heterocyclyl (e.g., heteroaryl), as defined herein, or any of the exemplary alkyl substituent groups described herein.

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkcycloalkyl, carboxyalkyl (e.g., optionally substituted with an O-protecting group, such as optionally substituted arylalkoxycarbonyl groups or any described herein), sulfoalkyl, acyl (e.g., acetyl, trifluoroacetyl, or others described herein), alkoxycarbonylalkyl (e.g., optionally substituted with an O-protecting group, such as optionally substituted arylalkoxycarbonyl groups or any described herein), heterocyclyl (e.g., heteroaryl), or alkheterocyclyl (e.g., alkheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. Amino groups can be unsubstituted amino (i.e., —NH₂) or substituted amino (i.e., —N(R^{N1})₂) groups. In a preferred embodiment, amino is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, carboxyalkyl, sulfoalkyl, acyl (e.g., acetyl, trifluoroacetyl, or others described herein), alkoxycarbonylalkyl (e.g., t-butoxycarbonylalkyl) or aryl, and each R^{N2} can be H, C₁₋₂₀ alkyl (e.g., C₁₋₆ alkyl), or C₆₋₁₀ aryl.

The term “amino acid,” as described herein, refers to a molecule having a side chain, an amino group, and an acid group (e.g., a carboxy group of —CO₂H or a sulfo group of —SO₃H), wherein the amino acid is attached to the parent molecular group by the side chain, amino group, or acid group (e.g., the side chain). In some embodiments, the amino acid is attached to the parent molecular group by a carbonyl group, where the side chain or amino group is attached to the carbonyl group. Exemplary side chains include an optionally substituted alkyl, aryl, heterocyclyl, alkaryl, alkheterocyclyl, aminoalkyl, carbamoylalkyl, and carboxyalkyl. Exemplary amino acids include alanine, arginine, asparagine, aspartic acid, cysteine, glutamic acid, glutamine, glycine, histidine, hydroxynorvaline, isoleucine, leucine, lysine, methionine, norvaline, ornithine, phenylalanine, proline, pyrrolysine, selenocysteine, serine, taurine, threonine, tryptophan, tyrosine, and valine. Amino acid groups may be optionally substituted with one, two, three, or, in the case of amino acid groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxy; (9) nitro; (10) oxo (e.g., carboxyaldehyde or acyl); (11) C₁₋₇ spirocyclyl; (12) thioalkoxy; (13) thiol; (14) —CO₂R^{A′}, where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alk-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alk-C₆₋₁₀ aryl, and (d) hydroxy; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alk-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR¹C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein.

The term “aryl,” as used herein, represents a mono-, bicyclic, or multicyclic carbocyclic ring system having one or two aromatic rings and is exemplified by phenyl, naphthyl, 1,2-dihydronaphthyl, 1,2,3,4-tetrahydronaphthyl, anthracenyl, phenanthrenyl, fluorenyl, indanyl, indenyl, and the like, and may be optionally substituted with 1, 2, 3, 4, or 5 substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₁₋₆ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) alkyl, (b) C₆₋₁₀ aryl, and (c) alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26) C₂₋₂₀ alkenyl; and (27) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The term “arylalkyl,” as used herein, represents an aryl group, as defined herein, attached to the parent molecular group through an alkylene group, as defined herein. Exemplary unsubstituted arylalkyl groups are from 7 to 30 carbons (e.g., from 7 to 16 or from 7 to 20 carbons, such as C₁₋₆ alk-C₆₋₁₀ aryl, C₁₋₁₀ alk-C₆₋₁₀ aryl, or C₁₋₂₀ alk-C₆₋₁₀ aryl). In some embodiments, the alkylene and the aryl each can be further substituted with 1, 2, 3, or 4 substituent groups as defined herein for the respective groups. Other groups preceded by the prefix “alk-” are defined in the same manner, where “alk” refers to a C₁₋₆ alkylene, unless otherwise noted, and the attached chemical structure is as defined herein.

The term “carbonyl,” as used herein, represents a C(O) group, which can also be represented as C═O.

The term “carboxy,” as used herein, means —CO₂H.

The term “cyano,” as used herein, represents an —CN group.

The term “cycloalkyl,” as used herein represents a monovalent saturated or unsaturated non-aromatic cyclic hydrocarbon group from three to eight carbons, unless otherwise specified, and is exemplified by cyclopropyl, cyclobutyl, cyclopentyl, cyclohexyl, cycloheptyl, bicycle heptyl, and the like. When the cycloalkyl group includes one carbon-carbon double bond or one carbon-carbon triple bond, the cycloalkyl group can be referred to as a “cycloalkenyl” or “cycloalkynyl” group respectively. Exemplary cycloalkenyl and cycloalkynyl groups include cyclopentenyl, cyclohexenyl, cyclohexynyl, and the like. Cycloalkyl groups can be optionally substituted with: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₆₋₁₀ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) C₆₋₁₀ alkyl, (b) C₆₋₁₀ aryl, and (c) C₁₋₆ alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₆₋₁₀ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26) oxo; (27) C₂₋₂₀ alkenyl; and (28) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The term “diastereomer,” as used herein means stereoisomers that are not mirror images of one another and are non-superimposable on one another.

The term “enantiomer,” as used herein, means each individual optically active form of a compound, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “halogen,” as used herein, represents a halogen selected from bromine, chlorine, iodine, or fluorine.

The term “heteroalkyl,” as used herein, refers to an alkyl group, as defined herein, in which one or two of the constituent carbon atoms have each been replaced by nitrogen, oxygen, or sulfur. In some embodiments, the heteroalkyl group can be further substituted with 1, 2, 3, or 4 substituent groups as described herein for alkyl groups. The terms “heteroalkenyl” and heteroalkynyl,” as used herein refer to alkenyl and alkynyl groups, as defined herein, respectively, in which one or two of the constituent carbon atoms have each been replaced by nitrogen, oxygen, or sulfur. In some embodiments, the heteroalkenyl and heteroalkynyl groups can be further substituted with 1, 2, 3, or 4 substituent groups as described herein for alkyl groups.

The term “heteroaryl,” as used herein, represents that subset of heterocyclyls, as defined herein, which are aromatic: i.e., they contain 4n+2 pi electrons within the mono- or multicyclic ring system. Exemplary unsubstituted heteroaryl groups are of 1 to 12 (e.g., 1 to 11, 1 to 10, 1 to 9, 2 to 12, 2 to 11, 2 to 10, or 2 to 9) carbons. In some embodiment, the heteroaryl is substituted with 1, 2, 3, or 4 substituents groups as defined for a heterocyclyl group.

The term “heteroarylalkyl,” as used here, refers to a heteroaryl group, as defined herein, attached to the parent molecular group through an alkylene group, as defined herein. Exemplary unsubstituted heteroarylalkyl groups are from 2 to 32 carbons (e.g., from 2 to 22, from 2 to 18, from 2 to 17, from 2 to 16, from 3 to 15, from 2 to 14, from 2 to 13, or from 2 to 12 carbons, such as C₁₋₆ alk-C₁₋₁₂ heteroaryl, C₁₋₁₀ alk-C₁₋₁₂ heteroaryl, or C₁₋₂₀ alk-C₁₋₁₂ heteroaryl). In some embodiments, the alkylene and the heteroaryl each can be further substituted with 1, 2, 3, or 4 substituent groups as defined herein for the respective group. Heteroarylalkyl groups are a subset of heterocyclylalkyl groups.

The term “heterocyclyl,” as used herein, represents a 5-, 6- or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocyclyl groups are of 1 to 12 (e.g., 1 to 11, 1 to 10, 1 to 9, 2 to 12, 2 to 11, 2 to 10, or 2 to 9) carbons. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like. Examples of fused heterocyclyls include tropanes and 1,2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, indazolyl, quinolyl, isoquinolyl, quinoxalinyl, dihydroquinoxalinyl, quinazolinyl, cinnolinyl, phthalazinyl, benzimidazolyl, benzothiazolyl, benzoxazolyl, benzothiadiazolyl, furyl, thienyl, thiazolidinyl, isothiazolyl, triazolyl, tetrazolyl, oxadiazolyl (e.g., 1,2,3-oxadiazolyl), purinyl, thiadiazolyl (e.g., 1,2,3-thiadiazolyl), tetrahydrofuranyl, dihydrofuranyl, tetrahydrothienyl, dihydrothienyl, dihydroindolyl, dihydroquinolyl, tetrahydroquinolyl, tetrahydroisoquinolyl, dihydroisoquinolyl, pyranyl, dihydropyranyl, dithiazolyl, benzofuranyl, isobenzofuranyl, benzothienyl, and the like, including dihydro and tetrahydro forms thereof, where one or more double bonds are reduced and replaced with hydrogens. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)- 1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2,3-dihydro-2-oxo-benzoxazolyl; 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7 H -purinyl); 1,2,3,6-tetrahydro-2,6-dioxo-1 H -purinyl (e.g., 1,2,3,6-tetrahydro-3,7-dimethyl-2,6-dioxo-1 H -purinyl); 2-oxobenz[c,d]indolyl; 1,1-dioxo-2H-naphth[1,8-c,d]isothiazolyl; and 1,8-naphthylenedicarboxamido. Additional heterocyclics include 3,3a,4,5,6,6a-hexahydro-pyrrolo[3,4-b]pyrrol-(2H)-yl, and 2,5-diazabicyclo[2.2.1]heptan-2-yl, homopiperazinyl (or diazepanyl), tetrahydropyranyl, dithiazolyl, benzofuranyl, benzothienyl, oxepanyl, thiepanyl, azocanyl, oxecanyl, and thiocanyl. Heterocyclic groups also include groups of the formula

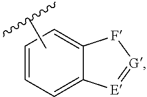

where E′ is selected from the group consisting of —N— and —CH—; F′ is selected from the group consisting of —N═CH—, —NH—CH₂—, —NH—C(O)—, —NH—, —CH═N—, —CH₂—NH—, —C(O)—NH—, —CH═CH—, —CH₂—, —CH₂CH₂—, —CH₂O—, —OCH₂—, —O—, and —S—; and G′ is selected from the group consisting of —CH— and —N—. Any of the heterocyclyl groups mentioned herein may be optionally substituted with one, two, three, four or five substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₂₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₁₋₆ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) C₁₋₆ alkyl, (b) C₆₋₁₀ aryl, and (c) C₁₋₆ alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) arylalkoxy; (25) C₁₋₆ alk-C₁₋₁₂ heterocyclyl (e.g., C₁₋₆ alk-C₁₋₁₂ heteroaryl); (26) oxo; (27) (C₁₋₁₂ heterocyclyl)imino; (28) C₂₋₂₀ alkenyl; and (29) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The term “hydrocarbon,” as used herein, represents a group consisting only of carbon and hydrogen atoms.

The term “hydroxyl,” as used herein, represents an —OH group. In some embodiments, the hydroxyl group can be substituted with 1, 2, 3, or 4 substituent groups (e.g., O-protecting groups) as defined herein for an alkyl.

The term “isomer,” as used herein, means any tautomer, stereoisomer, enantiomer, or diastereomer of any compound. It is recognized that the compounds can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). Unless otherwise noted, chemical structures depicted herein encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “N-protected amino,” as used herein, refers to an amino group, as defined herein, to which is attached one or two N-protecting groups, as defined herein.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999), which is incorporated herein by reference. N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups, such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “O-protecting group,” as used herein, represents those groups intended to protect an oxygen containing (e.g., phenol, hydroxyl, or carbonyl) group against undesirable reactions during synthetic procedures. Commonly used O-protecting groups are disclosed in Greene, “Protective Groups in Organic Synthesis,” 3^{rd} Edition (John Wiley & Sons, New York, 1999), which is incorporated herein by reference. Exemplary O-protecting groups include acyl, aryloyl, or carbamyl groups, such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl; alkylcarbonyl groups, such as acyl, acetyl, propionyl, pivaloyl, and the like; optionally substituted arylcarbonyl groups, such as benzoyl; silyl groups, such as trimethylsilyl (TMS), tert-butyldimethylsilyl (TBDMS), tri-iso-propylsilyloxymethyl (TOM), triisopropylsilyl (TIPS), and the like; ether-forming groups with the hydroxyl, such methyl, methoxymethyl, tetrahydropyranyl, benzyl, p-methoxybenzyl, trityl, and the like; alkoxycarbonyls, such as methoxycarbonyl, ethoxycarbonyl, isopropoxycarbonyl, n-isopropoxycarbonyl, n-butyloxycarbonyl, isobutyloxycarbonyl, sec-butyloxycarbonyl, t-butyloxycarbonyl, 2-ethylhexyloxycarbonyl, cyclohexyloxycarbonyl, methyloxycarbonyl, and the like; alkoxyalkoxycarbonyl groups, such as methoxymethoxycarbonyl, ethoxymethoxycarbonyl, 2-methoxyethoxycarbonyl, 2-ethoxyethoxycarbonyl, 2-butoxyethoxycarbonyl, 2-methoxyethoxymethoxycarbonyl, allyloxycarbonyl, propargyloxycarbonyl, 2-butenoxycarbonyl, 3-methyl-2-butenoxycarbonyl, and the like; haloalkoxycarbonyls, such as 2-chloroethoxycarbonyl, 2-chloroethoxycarbonyl, 2,2,2-trichloroethoxycarbonyl, and the like; optionally substituted arylalkoxycarbonyl groups, such as benzyloxycarbonyl, p-methylbenzyloxycarbonyl, p-methoxybenzyloxycarbonyl, p-nitrobenzyloxycarbonyl, 2,4-dinitrobenzyloxycarbonyl, 3,5-dimethylbenzyloxycarbonyl, p-chlorobenzyloxycarbonyl, p-bromobenzyloxy-carbonyl, fluorenylmethyloxycarbonyl, and the like; and optionally substituted aryloxycarbonyl groups, such as phenoxycarbonyl, p-nitrophenoxycarbonyl, o-nitrophenoxycarbonyl, 2,4-dinitrophenoxycarbonyl, p-methyl-phenoxycarbonyl, m-methylphenoxycarbonyl, o-bromophenoxycarbonyl, 3,5-dimethylphenoxycarbonyl, p-chlorophenoxycarbonyl, 2-chloro-4-nitrophenoxy-carbonyl, and the like); substituted alkyl, aryl, and alkaryl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl); carbonyl-protecting groups (e.g., acetal and ketal groups, such as dimethyl acetal, 1,3-dioxolane, and the like; acylal groups; and dithiane groups, such as 1,3-dithianes, 1,3-dithiolane, and the like); carboxylic acid-protecting groups (e.g., ester groups, such as methyl ester, benzyl ester, t-butyl ester, orthoesters, and the like; and oxazoline groups.

The term “oxo” as used herein, represents ═O.

The term “polyethylene glycol,” as used herein, represents an alkoxy chain comprised of one or more monomer units, each monomer unit consisting of —OCH₂CH₂—. Polyethyelene glycol (PEG) is also sometimes referred to as polyethylene oxide (PEO) or polyoxyethylene (POE), and these terms may be considered interchangeable for the purpose of this disclosure. For example, a polyethylene glycol may have the structure, —(CH₂)ₛ₂(OCH₂CH₂)ₛ₁(CH₂)ₛ₃O—, wherein s1 is an integer from 1 to 10 (e.g., from 1 to 6 or from 1 to 4), and each of s2 and s3, independently, is an integer from 0 to 10 (e.g., from 0 to 4, from 0 to 6, from 1 to 4, from 1 to 6, or from 1 to 10). Polyethylene glycol may also be considered to include an amino-polyethylene glycol of —NR^{N1}(CH₂)ₛ₂(CH₂CH₂O)ₛ₁(CH₂)ₛ₃NR^{N1}-, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds may exist in different tautomeric forms, all of the latter being included within the scope of the present disclosure.

The term “sulfonyl,” as used herein, represents an —S(O)₂— group.

The term “thiol,” as used herein represents an —SH group.

### Other Terms

As used herein, the term “about” or “approximately” refers to a ±10% variation from the recited quantitative value (and includes the recited quantitative value itself) unless otherwise indicated or inferred from the context. For example, unless otherwise state or inferred from the context, a dose of about 100 kBq/kg indicates a dose range of 100±10% kBq/kg, i.e., from 90 kBq/kg to 110 kBq/kg, inclusive.

As used herein, the term “administered in combination,” “combined administration,” or “co-administered” means that two or more agents are administered to a subject at the same time or within an interval such that there may be an overlap of an effect of each agent on the patient. Thus, two or more agents that are administered in combination need not be administered together. In some embodiments, they are administered within 90 days (e.g., within 80, 70, 60, 50, 40, 30, 20, 10, 5, 4, 3, 2, or 1 day(s)), within 28 days (e.g., with 14, 7, 6, 5, 4, 3, 2, or 1 day(s), within 24 hours (e.g., 12, 6, 5, 4, 3, 2, or 1 hour(s), or within about 60, 30, 15, 10, 5, or 1 minute of one another. In some embodiments, the administrations of the agents are spaced sufficiently closely together such that a combinatorial effect is achieved.

As used herein, “administering” an agent to a subject includes contacting cells of said subject with the agent.

As used herein, “antibody” refers to a polypeptide whose amino acid sequence including immunoglobulins and fragments thereof which specifically bind to a designated antigen, or fragments thereof. Antibodies may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acid sequences found in one or more regions of an antibody (e.g., variable region, hypervariable region, constant region, heavy chain, light chain, and combinations thereof) Moreover, those of ordinary skill in the art will appreciate that a characteristic sequence or portion of an antibody may include one or more polypeptide chains and may include sequence elements found in the same polypeptide chain or in different polypeptide chains.

As used herein, “antigen-binding fragment” refers to a portion of an antibody that retains the binding characteristics of the parent antibody.

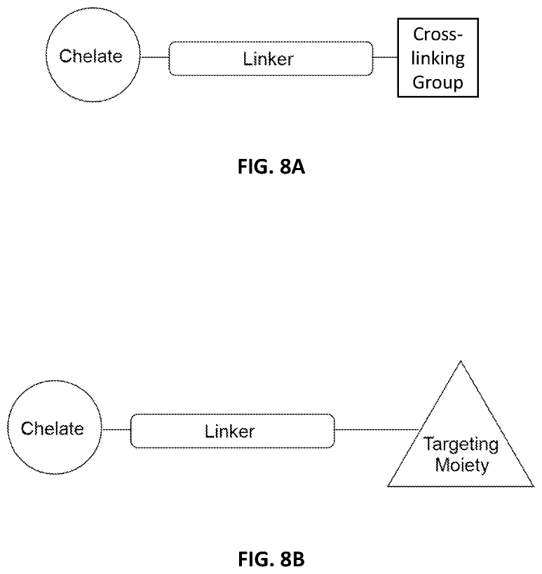

The term “bifunctional chelate,” as used herein, refer to a compound that comprises a chelate, a linker, and a cross-linking group. See, e.g., FIG. 8A. A “cross-linking group” is a reactive group that is capable of joining two or more molecules, e.g., joining a bifunctional chelate and a targeting moiety, by a covalent bond.

The term “bifunctional conjugate,” as used herein, refers to a compound that comprises a chelate or metal complex thereof, a linker, and a targeting moiety, e.g., an antibody or antigen-binding fragment thereof. See, e.g., FIG. 8B.

The term “cancer” refers to any cancer caused by the proliferation of malignant neoplastic cells, such as tumors, neoplasms, carcinomas, sarcomas, leukemias, and lymphomas. A “solid tumor cancer” is a cancer comprising an abnormal mass of tissue, e.g., sarcomas, carcinomas, and lymphomas. A “hematological cancer” or “liquid cancer,” as used interchangeably herein, is a cancer present in a body fluid, e.g., lymphomas and leukemias.

The term “checkpoint inhibitor,” also known as “immune checkpoint inhibitor” or “ICI,” refers to an agent which blocks the action of an immune checkpoint protein, e.g., blocks such immune checkpoint proteins from binding to their partner proteins.

The term “chelate,” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a therapeutic moiety, targeting moiety, or cross-linking group.

As used herein, the term “compound,” is meant to include all stereoisomers, geometric isomers, and tautomers of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H- 1,2,4-triazole, 1H- and 2H- isoindole, and 1H- and 2H-pyrazole. Tautomeric forms can be in equilibrium or sterically locked into one form by appropriate substitution.

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl. Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional.

The term “cross-linking group” as used herein refers to any reactive group that is able to join two or more molecules by a covalent bond. In some embodiments, the cross-linking group is an amino-reactive or thiol-reactive cross-linking group. In some embodiments, the amino-reactive or thiol-reactive cross-linking group comprises an activated ester such as a hydroxysuccinimide ester, 2,3,5,6-tetrafluorophenol ester, 4-nitrophenol ester or an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate. In some embodiments, the cross-linking group may be glycine-glycine-glycine and/or leucine-proline-(any amino acid)-threonine-glycine, which are the recognition sequences for coupling targeting agents with the linker using a sortase-mediated coupling reaction. The person having ordinary skill in the art will understand that the use of cross-linking groups are not limited to the specific constructs disclosed herein, but rather may include other known cross-linking groups.

As used herein, the terms “decrease,” “decreased,” “increase,” “increased,” or “reduction,” “reduced,” (e.g., in reference to therapeutic outcomes or effects) have meanings relative to a reference level. In some embodiments, the reference level is a level as determined by the use of said method with a control in an experimental animal model or clinical trial. In some embodiments, the reference level is a level in the same subject before or at the beginning of treatment. In some embodiments, the reference level is the average level in a population not being treated by said method of treatment.

The term an “effective amount” of an agent (e.g., any of the foregoing conjugates), as used herein, is that amount sufficient to effect beneficial or desired results, such as clinical results, and, as such, an “effective amount” depends upon the context in which it is being applied.

The term “immunoconjugate,” as used herein, refers to a conjugate that includes a targeting moiety (e.g., such as an antibody, nanobody, affibody, a consensus sequence from Fibronectin type III domain, a peptide, or a small molecule). In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 4, 5, or 8 conjugates per targeting moiety).

The term “lower effective dose,” when used as a term in conjunction with an agent (e.g., a therapeutic agent) refers to a dosage of the agent which is effective therapeutically in the combination therapies of the invention and which is lower than the dose which has been determined to be effective therapeutically when the agent is used as a monotherapy in reference experiments or by virtue of other therapeutic guidance.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

Compounds may have ionizable groups so as to be capable of preparation as pharmaceutically acceptable salts. These salts may be acid addition salts involving inorganic or organic acids or the salts may, in the case of acidic forms of compounds, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “polypeptide” as used herein refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

The term “radioconjugate,” as used herein, refers to any conjugate that includes a radioisotope or radionuclide, such as any of the radioisotopes or radionuclides described herein.

The term “radioimmunoconjugate,” as used herein, refers to any immunoconjugate that includes a radioisotope or radionuclide, such as any of the radioisotopes or radionuclides described herein.

The term “radioimmunotherapy,” as used herein, refers a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell, wherein administration of the radioimmunoconjugate kills the cell. Wherein radioimmunotherapy involves the selective killing of a cell, in some embodiments the cell is a cancer cell in a subject (e.g., a patient) having cancer.

As used herein, the term “radionuclide,” refers to an atom capable of undergoing radioactive decay (e.g., ³H, ⁴C, ¹⁵N, ¹⁸F, ³⁵S, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y ⁸⁷Y, ⁹⁰Y, ⁹⁷Ru, ⁹⁹Tc, ^{99m}Tc, ¹⁰⁵Rh, ¹⁰⁹Pd, ¹¹¹n, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ²²⁷Th, ^{229Th}, ⁶⁶Ga, ⁶⁷Ga, ⁶⁸Ga, ⁸²Rb, ^{117m}Sn, ²⁰¹Tl). The terms radioactive nuclide, radioisotope, or radioactive isotope may also be used to describe a radionuclide. Radionuclides may be used as detection agents, as described above. In some embodiments, the radionuclide is an alpha-emitting radionuclide.

By “subject” is meant a human (e.g., a patient) or non-human animal (e.g., a mammal).

By “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software, e.g., on the default setting (e.g., Sequence Analysis Software Package of the Genetics Computer Group, University of Wisconsin Biotechnology Center, 1710 University Avenue, Madison, WI 53705). Such software may match similar sequences by assigning degrees of homology to various substitutions, deletions, and other modifications.

The term “targeting moiety,” as used herein, refers to any molecule or any part of a molecule that binds to a given target. In some embodiments, the targeting moiety is a protein or polypeptide such as an antibody or antigen binding fragment thereof, a nanobody, an affibody, or a consensus sequence from a Fibronectin type III domain. In some embodiments, the targeting moiety is a peptide or a small molecule.

The term “therapeutic moiety,” as used herein, refers to any molecule or any part of a molecule that confers a therapeutic benefit. In some embodiments, the therapeutic moiety is a protein or polypeptide, e.g., an antibody, an antigen-binding fragment thereof. In some embodiments, the therapeutic moiety is a small molecule.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. In the context of cancer treatment, “ameliorating” may include, for example, reducing incidence of metastases, reducing tumor volume, reducing tumor vascularization and/or reducing the rate of tumor growth. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “tumor-associated antigen” means an antigen that is present on tumor cells at a significantly greater amount than on normal cells.

As used herein, the term “tumor-specific antigen” refers to an antigen that is endogenously present only on tumor cells.

### Radioimmunoconjugates

Radioimmunoconjugates suitable for use in accordance with the present disclosure generally refer to [²²⁵Ac]-radioimmunoconjugates, which comprises ²²⁵Ac chelated with a compound having the formula:

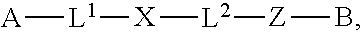

wherein each variable is as defined in the SUMMARY section above.

In some embodiments, the radioimmunoconjugate comprises the following structure:

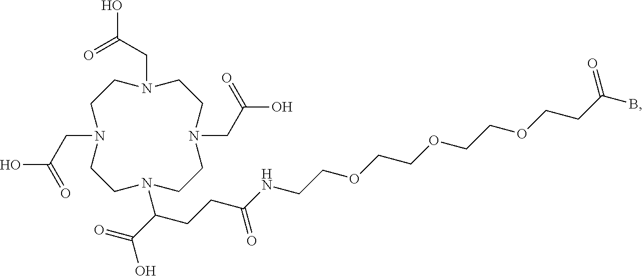

wherein B is a targeting moiety (e.g., an antibody or an antigen-binding fragment thereof, peptide, or small molecule).

In some embodiments, the radioimmunoconjugate comprises the following structure:

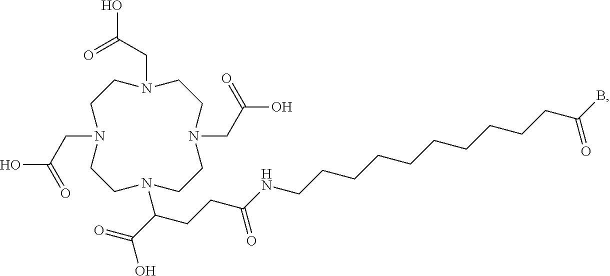

wherein B is a targeting moiety (e.g., an antibody or an antigen-binding fragment thereof, peptide, or small molecule).

### Antibodies and Antigen-Binding Fragments Thereof

Antibodies typically comprise two identical light polypeptide chains and two identical heavy polypeptide chains linked together by disulfide bonds. The first domain located at the amino terminus of each chain is variable in amino acid sequence, providing the antibody-binding specificities of each individual antibody. These are known as variable heavy (VH) and variable light (VL) regions. The other domains of each chain are relatively invariant in amino acid sequence and are known as constant heavy (CH) and constant light (CL) regions. Light chains typically comprise one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Antibodies described herein can include, for example, monoclonal antibodies, polyclonal antibodies, multispecific antibodies, human antibodies, humanized antibodies, camelid antibodies, chimeric antibodies, single-chain Fvs (scFv), disulfide-linked Fvs (sdFv), and anti-idiotypic (anti-Id) antibodies, and antigen-binding fragments of any of the above. In some embodiments, the antibody or antigen-binding fragment thereof is humanized. In some embodiments, the antibody or antigen-binding fragment thereof is chimeric. Antibodies can be of any type (e.g., IgG, IgE, IgM, IgD, IgA and IgY), class (e.g., IgG1, IgG2, IgG3, IgG4, IgA1 and IgA2) or subclass.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)₂ fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

Antibodies or fragments described herein can be produced by any method known in the art for the synthesis of antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using the methods described in, e.g., Morrison, 1985, Science 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods 248:1-6 (2001); and Tutt et al., J. Immunol. 147: 60 (1991).

In some embodiments, the antibody or antigen-binding fragment thereof is at least 100 kDa in size, e.g., at least 150 kDa in size, at least 200 kDa in size, at least 250 kDa in size, or at least 300 kDa in size.

### Insulin-Like Growth Factor 1 (IGF-1R) Antibodies

Insulin-like growth factor 1 receptor is a transmembrane protein found on the surface of human cells activated by insulin-like growth factor 1 (IGF-1) and 2 (IGF-2). In some embodiments, radioimmunoconjugates comprise antibodies against insulin-like growth factor-1 receptor (IGF-1R). Although not a typical oncogene, IGF-1R promotes initiation and progression of cancer, playing a critical role in mitogenic transformation and maintenance of the transformed phenotype. IGF-1R has been associated with development of multiple common cancers including breast, lung (e.g., non-small lung), liver, prostate, pancreas, ovarian, colon, melanoma, adrenocortical carcinoma, and various types of sarcomas. IGF-1R signaling stimulates tumour cell proliferation and metabolism, supports angiogenesis, and confers protection from apoptosis. It affects metastatic factors (e.g., HIF-1 dependent hypoxia signaling), anchorage independent growth, as well as growth and survival of tumour metastases after extravasation. IGF-1R has also been implicated in the development, maintenance and enrichment of therapeutic resistant cancer stem cell populations.

Despite the abundance of data implicating IGF-1R's role in cancer, therapeutics targeting IGF-1R have yet to demonstrate a significant impact on disease. There has been much speculation for this lack of efficacy including the inability to identify appropriate biomarkers for patient identification, complexity and interdependency of the IGF-1/IR signaling pathway and the development of other growth hormone compensatory mechanisms [Beckwith and Yee, Mol Endocrinol, November 2015, 29(11):1549-1557]. Radioimmunotherapy, however, may provide a viable mechanism for treating cancers overexpressing the IGF-1 receptor by utilizing the ability of IGF-1R to undergo antibody triggered internalization and lysosomal degradation to deliver targeted radioisotopes inside cancer cells. Internalization and lysosomal degradation of an IGF-1R targeted radioimmunoconjugate prolongs the residence time of the delivered radioisotope inside cancer cells, thereby maximizing the potential for a cell killing emission to occur. In the case of actinium-225, which yields 4 alpha particles per decay chain, cell death can be accomplished by as little as 1 atom of radionuclide delivered per cell [Sgouros, et al. J Nucl Med. 2010, 51:311-2]. Cell killing due to direct DNA impact and breakage by an alpha particle may occur in the targeted cell or in a radius of 2 or 3 non-targeted cells for a given alpha particle decay. In addition to having very high potential anti-tumour potency, IGF-1R targeted radioimmunoconjugates may not generate mechanistic resistance as they do not rely on blocking ligand binding to the receptor to inhibit the oncologic process, as needed with a therapeutic antibody.

Several IGF-1R antibodies have been developed and investigated for the treatment of various types of cancers, including figitumumab, cixutumumab, TAB-199, AVE1642 (also known as humanized EM164 and huEM164), BIIB002, robatumumab, and teprotumumab. After binding to IGF-1R, these antibodies are internalized into the cell and degraded by lysosomal enzymes. The combination of overexpression on tumor cells and internalization offers the possibility of delivering detection agents directly to the tumor site while limiting the exposure of normal tissues to toxic agents.

In some embodiments, the light chain variable region of the IGF-1R antibody or antibody-binding fragment thereof comprises one, two, or three complementarity determining regions (CDRs) CDR-L1, CDR-L2, and/or CDR-L3, with amino acid sequences of AVE1642 as shown below, or CDR region(s) having an amino acid sequence(s) differing in 1 or 2 amino acids therefrom:

The CDRs of the light chain variable region of AVE1642 comprises the sequences:

(CDR-L1)
SEQ ID NO: 1
RSSQSIVHSNVNTYLE
(CDR-L2)
SEQ ID NO: 2
KVSNRFS
(CDR-L3)
SEQ ID NO: 3
FQGSHVPPT

In some embodiments, the light chain variable region of the IGF-1R antibody or antigen-binding fragment thereof comprises the light chain variable region of AVE1642 (SEQ ID NO: 4) or an amino acid sequence differing in 1, 2, 3, or 4 amino acids therefrom, or an amino acid sequence having at least 85%, at least 90%, at least 95%, at least 97%, or at least 99% identical to the light chain variable region of AVE1642 (SEQ ID NO: 4):

The light chain variable region of AVE1642 comprises the sequence:

SEQ ID NO: 4
DVVMTQTPLSLPVSLGDPASISCRSSQSIVHSNVNTYLEWYLQKPGQSP
RLLIYKVSNRFSGVPDRFSGSGAGTDFTLRISRVEAEDLGIYYCFQGSH
VPPTFGGGTKLEIKR

In some embodiments, the heavy chain variable region of the IGF-1R antibody or antibody-binding fragment thereof comprises one, two, or three complementarity determining regions (CDRs) CDR-H1, CDR-H2, and/or CDR-H3, with amino acid sequences of AVE1642 as shown below, or CDR region(s) having an amino acid sequence(s) differing in 1 or 2 amino acids therefrom:

The CDRs of the heavy chain variable region of AVE1642 comprise the sequences:

(CDR-H1)
SEQ ID NO: 5
SYWMH
(CDR-H2)
SEQ ID NO: 6
EINPSNGRTNYNQKFQG
(CDR-H3)
SEQ ID NO: 7
GRPDYYGSSKWYFDV

In some embodiments, the heavy chain variable region of the IGF-1R antibody or antigen-binding fragment thereof comprises the heavy chain variable region of AVE1642 (SEQ ID NO: 8) or an amino acid sequence differing in 1, 2, 3, or 4 amino acids therefrom, or an amino acid sequence having at least 85%, at least 90%, at least 95%, at least 97%, or at least 99% identical to the heavy chain variable region of AVE1642 (SEQ ID NO: 8):

The heavy chain variable region of AVE1642 comprises the sequence:

SEQ ID NO: 8
QVQLVQSGAEVVKPGASVKLSCKASGYTFTSYWMHWVKQRPGQGLEWIG
EINPSNGRTNYNQKFQGKATLTVDKSSSTAYMQLSSLTSEDSAVYYFAR
GRPDYYGSSKWYFDVWGQGTTVTVSS

The light chain of AVE1642 comprises the sequence

SEQ ID NO: 22
DVVMTQTPLSLPVSLGDPASISCRSSQSIVHSNVNTYLEWYLQKPGQSP
RLLIYKVSNRFSGVPDRFSGSGAGTDFTLRISRVEAEDLGIYYCFQGSH
VPPTFGGGTKLEIKRTVAAPSVFIFPPSDEQLKSGTASVVCLLNNFYPR
EAK

The heavy chain of AVE1642 comprises the sequence

SEQ ID NO: 23
QVQLVQSGAEVVKPGASVKLSCKASGYTFTSYWMHWVKQRPGQGLEWIG
EINPSNGRTNYNQKFQGKATLTVDKSSSTAYMQLSSLTSEDSAVYYFAR
GRPDYYGSSKWYFDVWGQGTTVTVSSASTKGPSVFPLAPSSKSTSGGTA
ALG

### Endosialin (TEM-1) Antibodies

Endosialin, also known as TEM-1 or CD-248, is an antigen expressed by tumor-associated endothelial cells, stromal cells, and pericytes.

Examples of endosialin antibodies include hMP-E-8.3 (disclosed in WO 2017/134234, the entire contents of which are incorporated by reference herein) and ontuxizumab (MORAb-004).

### Fibroblast Growth Factor Receptor 3 (FGFR3) Antibodies

Fibroblast growth factor receptor 3 (FGFR3) plays critical roles during embryonic development, tissue homeostasis and metabolism, by regulating a broad array of cellular processes, including proliferation, differentiation, migration and survival, in a context-dependent manner. It is overexpressed in many cancer types, often due to mutations that confer constitutive activation.

In some embodiments, provided methods employ an [²²⁵Ac]-radioimmunoconjugate that comprises an antibody, or an antigen-binding fragment thereof, targeting FGFR3.

In certain embodiments, amino acid sequence variants of antibodies or antigen-binding fragments thereof are contemplated; e.g., variants that are capable of binding to human FGFR3 and/or a mutant FGFR3 (such as a mutant FGFR3 associated with cancer). For example, it may be desirable to improve the binding affinity and/or other biological properties of the antibody or antigen-binding fragment thereof. Amino acid sequence variants of an antibody or antigen-binding fragment thereof may be prepared by introducing appropriate modifications into the nucleotide sequence encoding the antibody or antigen-binding fragment thereof, or by peptide synthesis. Such modifications include, for example, deletions from and/or insertions into and/or substitutions of residues within the amino acid sequences of the antibody or antigen-binding fragment thereof. Any combination of deletion, insertion and substitution can be made to arrive at the final construct, provided that the final construct possesses desired characteristics, e.g. antigen binding.

In some embodiments, the antibody or antigen binding fragment thereof is an inhibitory antibody (also called “antagonistic antibody”) or antigen-binding fragment thereof, e.g., the antibody or antigen binding fragment thereof at least partially inhibits one or more functions of the target molecule (e.g., FGFR3) as explained further herein.

Non-limiting examples of inhibitory antibodies include humanized monoclonal antibodies such as MFGR1877S (CAS No. 1312305-12-6; Genentech) (a human monoclonal antibody also known as vofatamab, and whose lyophilized form is also known as B-701 or R3Mab); PRO-001 (Prochon); PRO-007 (Fibron); IMC-D11 (Imclone); and AV-370 (Aveo Pharmaceuticals). (See, e.g., U.S. Pat. Nos. 8,410,250; 10,208,120; and International Patent Publication Nos. WO2002102972A2, WO2002102973A2, WO2007144893A2, WO2010002862A2, and WO2010048026A2.)

In some embodiments, the antibody or antigen binding fragment thereof is an agonistic antibody (also known as stimulatory antibody).

In some embodiments, the antibody or antigen biding fragment thereof is neither agonistic or antagonistic, or has not been characterized as either agonistic or antagonistic.

Additional known FGFR3 antibodies include, for example, mouse monoclonal antibodies such as, for example, 1G6, 6G1, and 15B2 from Genentech (See, e.g., U.S. Pat. No. 8,410,250), B9 (Sc-13121) (Santa Cruz Biotechnology), MAB766 (clone 136334) (R&D systems), MAB7661 (clone 136318) (R&D systems), and OTI1B10 (OriGene); rabbit polyclonal antibodies such as, for example, ab10651 (Abcam); and rabbit monoclonal antibodies such as C51F2 (catalog number 4574) (Cell Signaling Technology).

In certain embodiments of the present disclosure, the antibody or antigen-binding fragment thereof comprises specific heavy chain complementarity determining regions CDR-H1, CDR-H2 and/or CDR-H3 as described herein. In some embodiments, the complementarity determining regions (CDRs) of the antibody or antigen-binding fragment thereof are flanked by framework regions. A heavy or light chain of an antibody or antigen-binding fragment thereof containing three CDRs typically contains four framework regions.

In some embodiments, the heavy chain variable region of the FGFR3 antibody or antibody-binding fragment thereof comprises one, two, or three complementarity determining regions (CDRs) CDR-H1, CDR-H2, and/or CDR-H3, with amino acid sequences shown below, or CDR region(s) having an amino acid sequence(s) differing in 1 or 2 amino acids therefrom:

CDR-H1:
(SEQ ID NO: 9)
GFTFTSTGIS
CDR-H2:
(SEQ ID NO: 10)
GRIYPTSGSTNYADSV
CDR-H3:
(SEQ ID NO: 11)
TYGIYDLYVDYTEYVMDY
or
(SEQ ID NO: 12)
ARTYGIYDLYVDYTEYVMDY

In some embodiments, the light chain variable region of the FGFR3 antibody or antibody-binding fragment thereof comprises one, two, or three complementarity determining regions (CDRs) CDR-L1, CDR-L2, and/or CDR-L3. with amino acid sequences as shown below, or CDR region(s) having an amino acid sequence(s) differing in 1 or 2 amino acids therefrom:

CDR-L1:
(SEQ ID NO: 13)
RASQDVDTSLA
CDR-L2:
(SEQ ID NO: 14)
SASFLYS
CDR-L3:
(SEQ ID NO: 15)
QQSTGHPQT

In some embodiments, the antibody or antigen-binding fragment thereof has CDR sequences having amino acid sequences of SEQ ID NOs: 9, 10, 11, 13, 14, and 15 without any variation. For example, in some embodiments, the antibody or antigen-binding fragment thereof comprises heavy chain complementary determining regions CDR-H1, CDR-H2, and CDR-H3 having the amino acid sequences of SEQ ID NOs: 9, 10, and 11, and the chain complementarity determining regions CDR-L1, CDR-L2, and CDR-L3 having the amino acid sequences of SEQ ID NOs: 13, 14, and 15.

In some embodiments, the antibody or antigen-binding fragment thereof has CDR sequences having amino acid sequences of SEQ ID NOs: 9, 10, 12, 13, 14, and 15 without any variation. For example, in some embodiments, the antibody or antigen-binding fragment thereof comprises heavy chain complementary determining regions CDR-H1, CDR-H2, and CDR-H3 having the amino acid sequences of SEQ ID NOs: 9, 10, and 12, and the chain complementarity determining regions CDR-L1, CDR-L2, and CDR-L3 having the amino acid sequences of SEQ ID NOs: 13, 14, and 15.

In some embodiments, the heavy chain variable region of the FGFR3 antibody or antigen-binding fragment thereof comprises an amino acid sequence of SEQ ID NO: 16 or an amino acid sequence differing in 1, 2, 3, or 4 amino acids therefrom, or an amino acid sequence having at least 85%, at least 90%, at least 95%, at least 97%, or at least 99% identical to SEQ ID NO: 16:

(SEQ ID NO: 16)
EVQLVESGGG LVQPGGSLRL SCAASGFTFT STGISWVRQA
PGKGLEWVGR IYPTSGSTNY ADSVKGRFTI SADTSKNTAY
LQMNSLRAED TAVYYCARTY GIYDLYVDYT EYVMDYWGQG TLV

In some embodiments, the heavy chain variable region of the FGFR3 antibody or antigen-binding fragment thereof comprises an amino acid sequence of SEQ ID NO: 18 or an amino acid sequence differing in 1, 2, 3, or 4 amino acids therefrom, or an amino acid sequence having at least 85%, at least 90%, at least 95%, at least 97%, or at least 99% identical to SEQ ID NO: 18:

(SEQ ID NO: 18)
EVQLVESGGG LVQPGGSLRL SCAASGFTFT STGISWVRQA
PGKGLEWVGR IYPTSGSTNY ADSVKGRFTI SADTSKNTAY
LQMNSLRAED TAVYYCARAR TYGIYDLYVD YTEYVMDYWG
QGTLV

In some embodiments, the heavy chain of the FGFR3 antibody comprises a constant region having an amino acid sequence of SEQ ID NO: 20 or an amino acid sequence differing in 1, 2, 3, or 4 amino acids therefrom, or an amino acid sequence having at least 85%, at least 90%, at least 95%, at least 97%, or at least 99% identical to SEQ ID NO: 20:

(SEQ ID NO: 20)
ASTKGP SVFPLAPSSK STSGGTAALG CLVKDYFPEP VTVSWNSGAL
TSGVHTFPAV LQSSGLYSLS SVVTVPSSSL GTQTYICNVN
HKPSNTKVDK KVEPKSCDKT HTCPPCPAPE LLGGPSVELF
PPKPKDTLMI SRTPEVTCVV VDVSHEDPEV KFNWYVDGVE
VHNAKTKPRE EQYNSTYRVV SVLTVLHQDW LNGKEYKCKV
SNKALPAPIE KTISKAKGQP REPQVYTLPP SREEMTKNQV
SLTCLVKGFY PSDIAVEWES NGQPENNYKT TPPVLDSDGS
FFLYSKLTVD KSRWQQGNVF SCSVMHEALH NHYTQKSLSL SPGK

In some embodiments, the heavy chain of the FGFR3 antibody comprises a sequence having an amino acid sequence of SEQ ID NO: 24 or an amino acid sequence differing in 1, 2, 3, or 4 amino acids therefrom, or an amino acid sequence having at least 85%, at least 90%, at least 95%, at least 97%, or at least 99% identical to SEQ ID NO: 24:

(SEQ ID NO: 24)
TVSSASTKGP SVFPLAPSSK STSGGTAALG CLVKDYFPEP
VTVSWNSGAL TSGVHTFPAV LQSSGLYSLS SVVTVPSSSL
GTQTYICNVN HKPSNTKVDK KVEPKSCDKT HTCPPCPAPE
LLGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SREEMTKNQV SLTCLVKGFY PSDIAVEWES NGQPENNYKT
TPPVLDSDGS FFLYSKLTVD KSRWQQGNVF SCSVMHEALH
NHYTQKSLSL SPGK

In some embodiments, the light chain variable region of the FGFR3 antibody or antigen-binding fragment thereof comprises an amino acid sequence of SEQ ID NO: 17 or an amino acid sequence differing in 1, 2, 3, or 4 amino acids therefrom, or an amino acid sequence having at least 85%, at least 90%, at least 95%, at least 97%, or at least 99% identical to SEQ ID NO: 17:

(SEQ ID NO: 17)
DIQMTQSPSS LSASVGDRVT ITCRASQDVD TSLAWYKQKP
GKAPKLLIYS ASFLYSGVPS RFSGSGSGTD FTLTISSLQP
EDFATYYCQQ STGHPQTFGQ GTKVEIK

In some embodiments, the light chain variable region of the FGFR3 antibody or antigen-binding fragment thereof comprises an amino acid sequence of SEQ ID NO: 19 or an amino acid sequence differing in 1, 2, 3, or 4 amino acids therefrom, or an amino acid sequence having at least 85%, at least 90%, at least 95%, at least 97%, or at least 99% identical to SEQ ID NO: 19:

(SEQ ID NO: 19)
DIQMTQSPSS LSASVGDRVT ITCRASQDVD TSLAWYQQKP
GKAPKLLIYS ASFLYSGVPS RFSGSGSGTD FTLTISSLQP
EDFATYYCQQ STGHPQTFGQ GTKVEIK

In some embodiments, the light chain of the FGFR3 antibody comprises a constant region having an amino acid sequence of SEQ ID NO: 21 or an amino acid sequence differing in 1, 2, 3, or 4 amino acids therefrom, or an amino acid sequence having at least 85%, at least 90%, at least 95%, at least 97%, or at least 99% identical to SEQ ID NO: 21:

(SEQ ID NO: 21)
RTVAAPSVFI FPPSDEQLKS GTASVVCLLN NFYPREAKVQ
WKVDNALQSG NSQESVTEQD SKDSTYSLSS TLILSKADYE
KHKVYACEVT HQGLSSPVTK SFNRGEC

In some embodiments, the FGFR3 antibody or antigen-binding fragment thereof comprises at least one, two, three, four, five, or six complementarity determining regions (CDRs) selected from the group consisting of:
CDR-H1 comprising the amino acid sequence of SEQ ID NO: 9, or an amino acid sequence differing in 1 or 2 amino acids therefrom;
CDR-H2 comprising the amino acid sequence of SEQ ID NO: 10, or an amino acid sequence differing in 1 or 2 amino acids therefrom;
CDR-H3 comprising the amino acid sequence of SEQ ID NO: 11 or 12, or an amino acid sequence differing in 1 or 2 amino acids from SEQ ID NO: 11 or 12;
CDR-L1 comprising the amino acid sequence of SEQ ID NO: 13, or an amino acid sequence differing in 1 or 2 amino acids therefrom;
CDR-L2 comprising the amino acid sequence of SEQ ID NO: 14, or an amino acid sequence differing in 1 or 2 amino acids therefrom; and
CDR-L3 comprising the amino acid sequence of SEQ ID NO: 15, or an amino acid sequence differing in 1 or 2 amino acids therefrom.

In some embodiments, the antibody or antigen-binding fragment thereof comprises: (i) a heavy chain variable domain comprising the amino acid sequence of SEQ ID NO: 16 or SEQ ID NO: 18; and (ii) a light chain variable domain comprising the amino acid sequence of SEQ ID NO: 17 or SEQ ID NO: 19.

In some embodiments, the antibody or antigen-binding fragment thereof comprises: (i) a heavy chain variable domain comprising the amino acid sequence of SEQ ID NO: 16; and (ii) a light chain variable domain comprising the amino acid sequence of SEQ ID NO: 17.

In some embodiments, the antibody or antigen-binding fragment thereof comprises: (i) a heavy chain variable domain comprising the amino acid sequence of SEQ ID NO: 18; and (ii) a light chain variable domain comprising the amino acid sequence of SEQ ID NO: 19.

In some embodiments, the FGFR3 antibody is MFGR1877S (vofatamab).

### Nanobodies

Nanobodies are antibody fragments consisting of a single monomeric variable antibody domain. Nanobodies may also be referred to as single-domain antibodies. Like antibodies, nanobodies bind selectively to a specific antigen. Nanobodies may be heavy-chain variable domains or light chain domains. Nanobodies may occur naturally or be the product of biological engineering. Nanobodies may be biologically engineered by site-directed mutagenesis or mutagenic screening (e.g., phage display, yeast display, bacterial display, mRNA display, ribosome display).

### Affibodies

Affibodies are polypeptides or proteins engineered to bind to a specific antigen. As such, affibodies may be considered to mimic certain functions of antibodies. Affibodies may be engineered variants of the B-domain in the immunoglobulin-binding region of staphylococcal protein A. Affibodies may be engineered variants of the Z-domain, a B-domain that has lower affinity for the Fab region. Affibodies may be biologically engineered by site-directed mutagenesis or mutagenic screening (e.g., phage display, yeast display, bacterial display, mRNA display, ribosome display).

Affibody molecules showing specific binding to a variety of different proteins (e.g. insulin, fibrinogen, transferrin, tumor necrosis factor-α, IL-8, gp120, CD28, human serum albumin, IgA, IgE, IgM, HER2 and EGFR) have been generated, demonstrating affinities (K_{d}) in the μM to pM range.

### Fibronectin Type III Domains

The Fibronectin type III domain is an evolutionarily conserved protein domain found in a wide-variety of extracellular proteins. The Fibronectin type III domain has been used as a molecular scaffold to produce molecules capable of selectively binding a specific antigen. Variants of the Fibronectin type III domains (FN3) that have been engineered for selective-binding may also be referred to as monobodies. FN3 domains may be biologically engineered by site-directed mutagenesis or mutagenic screening (e.g., CIS-display, phage display, yeast display, bacterial display, mRNA display, ribosome display).

### Modified Polypeptides

Polypeptides used in accordance with the disclosure may have a modified amino acid sequence. Modified polypeptides may be substantially identical to the corresponding reference polypeptide (e.g., the amino acid sequence of the modified polypeptide may have at least 50%, 60%, 70%, 75%, 80%, 85%, 90%, 95%, 96%, 97%, 98%, 99%, or 100% identity to the amino acid sequence of the reference polypeptide). In certain embodiments, the modification does not destroy significantly a desired biological activity (e.g., binding to IGF-1R or to endosialin). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified polypeptide may have or may optimize a characteristic of a polypeptide, such as in vivo stability, bioavailability, toxicity, immunological activity, immunological identity, and conjugation properties.

Modifications include those by natural processes, such as post-translational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from post-translational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy-terminus of a polypeptide can facilitate conjugation of these polypeptides by, e.g., disulfide bonding. For example, a polypeptide can be modified to include a single cysteine residue at the amino-terminus or a single cysteine residue at the carboxy-terminus. Amino acid substitutions can be conservative (i.e., wherein a residue is replaced by another of the same general type or group) or non-conservative (i.e., wherein a residue is replaced by an amino acid of another type). In addition, a naturally occurring amino acid can be substituted for a non-naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, N-protected amino acids, an amino acid having an acetylaminomethyl group attached to a sulfur atom of a cysteine, a pegylated amino acid, the omega amino acids of the formula NH2(CH2)nCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 1. If such substitutions result in a change not desired, then other type of substitutions, denominated “exemplary substitutions” in Table 1, or as further described herein in reference to amino acid classes, are introduced and the products screened.

TABLE 1
Amino acid substitutions

| Original | | Conservative |
|---|---|---|
| residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Substantial modifications in function or immunological identity are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

### Chelating Moieties

Examples of suitable chelating moieties include, but are not limited to, DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N″′, N″″-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), H₆phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N″′, N″″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), HOPO (octadentate hydroxypyridinones), or porphyrins.

Preferably, the chelating moiety is selected from DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (10-(2-hydroxypropyl)-1,4,7-tetraazacyclododecane-1,4,7-triacetic acid).

In some embodiments, the chelating moiety is DOTA.

In some embodiments, chelating moieties are useful as detection agents, and radioimmunoconjugates comprising such detectable chelating moieties can therefore be used as diagnostic or theranostic agents.

### Linkers

Referring to the [²²⁵Ac]-radioimmunoconjugate, the linker is as shown within the structure of the following formula:

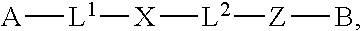

wherein the linker typically includes -L¹-X-L²-Z—, in which:
L¹ is a bond or optionally substituted C₁₋₆ alkyl or C₁₋₆ heteroalkyl;
X is —C(O)NR¹—*, —NR¹C(O)—*, —OC(O)NR¹—*, —NR¹C(O)O—*, —NR¹C(O)NR¹—, —CH₂-Ph-C(O)NR¹—*, —NR¹C(O)-Ph-CH₂—*, —O—, or —NR¹—, wherein “*” indicates the attachment point to L², and each R¹ is independently hydrogen or C₁₋₆ alkyl;
L² is optionally substituted C₁₋₅₀ alkyl or C₁₋₅₀ heteroalkyl;
Z is —C(O)—, —CH₂—, —OC(O)—#, —C(O)O—#, —NR²C(O)—#, —C(O)NR²—#, or —NR²—, wherein “#” indicates the attachment point to B, and each R² is independently hydrogen or C₁₋₆ alkyl.

In some embodiments, L¹ is optionally substituted C₁₋₆ alkyl. For example, L¹ is —CH₂CH₂—. For example, L¹ has the structure:

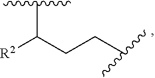

wherein R² is hydrogen or —CO₂H.

In some embodiments, X is —C(O)NR¹—*, “*” indicating the attachment point to L², and R¹ is H.

In some embodiments, L² is optionally substituted C₁₋₅₀ alkyl (e.g., C₁₋₄₀ alkyl, C₁₋₃₀ alkyl, C₁₋₂₀ alkyl, C₂₋₁₈ alkyl, C₃₋₁₆ alkyl, C₄₋₁₄ alkyl, C₅₋₁₂ alkyl, C₆₋₁₀ alkyl, C₈₋₁₀ alkyl, or C₁₀ alkyl). For example, L² is C₁₀ alkyl as shown below:

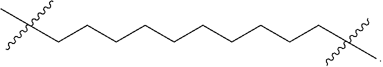 [SMILES: *CCCCCCCCCC*]

In some embodiments, L² is optionally substituted C₁₋₅₀ heteroalkyl (e.g., C₁₋₄₀ heteroalkyl, C₁₋₃₀ heteroalkyl, C₁₋₂₀ heteroalkyl, C₂₋₁₈ heteroalkyl, C₃₋₁₆ heteroalkyl, C₄₋₁₄ heteroalkyl, C₅₋₁₂ heteroalkyl, C₆₋₁₀ heteroalkyl, C₈₋₁₀ heteroalkyl, C₄ heteroalkyl, C₆ heteroalkyl, C₈ heteroalkyl, C₁₀ heteroalkyl, C₁₂ heteroalkyl, C₁₆ heteroalkyl, C₂₀ heteroalkyl, or C₂₄ heteroalkyl). In certain embodiments, L² is optionally substituted C₁₋₅₀ heteroalkyl comprising a polyethylene glycol (PEG) moiety comprising 1-20 oxyethylene (—O—CH₂—CH₂—) units, e.g., 2 oxyethylene units (PEG2), 3 oxyethylene units (PEG3), 4 oxyethylene units (PEG4), 5 oxyethylene units (PEG5), 6 oxyethylene units (PEG6), 7 oxyethylene units (PEG7), 8 oxyethylene units (PEG8), 9 oxyethylene units (PEG9), 10 oxyethylene units (PEG10), 12 oxyethylene units (PEG12), 14 oxyethylene units (PEG14), 16 oxyethylene units (PEG16), or 18 oxyethylene units (PEG18).

In certain embodiments, L² is optionally substituted C₁₋₅₀ heteroalkyl comprising a polyethylene glycol (PEG) moiety comprising 1-20 oxyethylene (—O—CH₂—CH₂—) units or portions thereof. For example, L² is a linker comprising PEG3 as shown below:

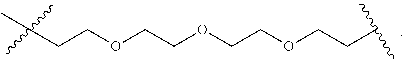 [SMILES: *CCOCCOCCOCC*]

In some embodiments, Z is —C(O)— or —CH₂—. In some embodiments, Z is —C(O)— and is the point of conjugation to B via a lysine residue from B.

In certain embodiments, the formula A-L¹-X-L²-Z—B can be represented by the following structure:

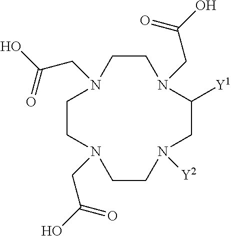

wherein Y¹ is —CH₂OCH₂L²-B, C(O)L²-B, or C(S)L²-B and Y² is —CH₂CO₂H; or Y¹ is H and Y² is L¹-X-L²-B.

### Checkpoint Inhibitors

In some embodiments, a checkpoint inhibitor is co-administered with a radioimmunoconjugate. Generally, suitable checkpoint inhibitors inhibit an immune suppressive checkpoint protein. In some embodiments, the checkpoint inhibitor inhibits a protein selected from the group consisting of cytotoxic T-lymphocyte-associated antigen 4 (CTLA-4), programmed death 1 (PD-1), programmed death ligand-1 (PD-L1), LAG-3, T cell immunoglobulin mucin 3 (TIM-3), and killer immunoglobulin-like receptors (KIRs).

For example, in some embodiments, the checkpoint inhibitor is capable of binding to CTLA-4, PD-1, or PD-L1. In some embodiments, the checkpoint inhibitor interferes with the interaction (e.g., interferes with binding) between PD-1 and PD-L1.

In some embodiments, the checkpoint inhibitor is a small molecule.

In some embodiments, the checkpoint inhibitor is an antibody or antigen-binding fragment thereof, e.g., a monoclonal antibody. In some embodiments, the checkpoint inhibitor is a human or humanized antibody or antigen-binding fragment thereof. In some embodiments, the checkpoint inhibitor is a mouse antibody or antigen-binding fragment thereof.

In some embodiments, the checkpoint inhibitor is a CTLA-4 antibody. Non-limiting examples of CTLA-4 antibodies include BMS-986218, BMS-986249, ipilimumab, tremelimumab (formerly ticilimumab, CP-675,206), MK-1308, and REGN-4659. An additional example of a CTLA-4 antibody is 4F10-11, a mouse monoclonal antibody.

In some embodiments, the checkpoint inhibitor is a PD-1 antibody. Non-limiting examples of PD-1 antibodies include camrelizumab, cemiplumab, nivolumab, pembrolizumab, sintilimab, tislelizumab and toripalimab. An additional example of a PD-1 antibody is RMP1-14, a mouse monoclonal antibody. In some embodiments, the checkpoint inhibitor is pembrolizumab.

In some embodiments, the checkpoint inhibitor is a PD-L1 antibody. Non-limiting examples of PD-L1 antibodies include atezolizumab, avelumab, and durvalumab.

In some embodiments, a combination of more than one checkpoint inhibitor is used. For example, in some embodiments, both a CTLA-4 inhibitor and a PD-1 or PD-L1 inhibitor is used.

### Combination Therapies

As provided above, the present disclosure relates to combination therapies comprising [²²⁵Ac]-radioimmunoconjugates and one or more checkpoint inhibitors for effective treatment of cancers at certain dose levels.

In some embodiments, the combination therapy comprises a PD-1 inhibitor or a CTLA-4 inhibitor, and an [²²⁵Ac]-radioimmunoconjugate that comprises ²²⁵Ac chelated with one of the following compounds:

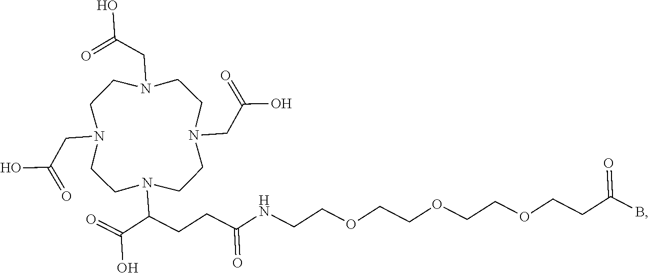

B being an IGF-1R antibody or an antigen-binding domain thereof,

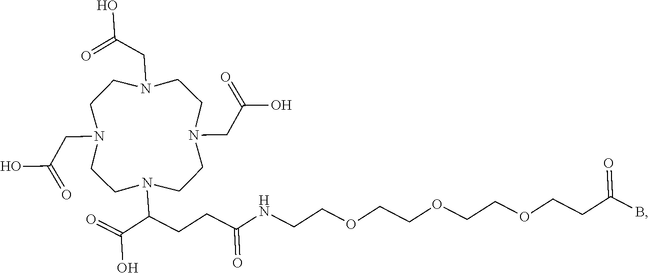

B being an FGFR3 antibody or an antigen-binding domain thereof,

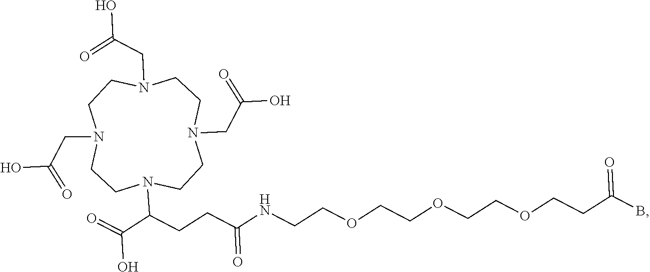

B being a TEM-1 antibody or an antigen-binding domain thereof,

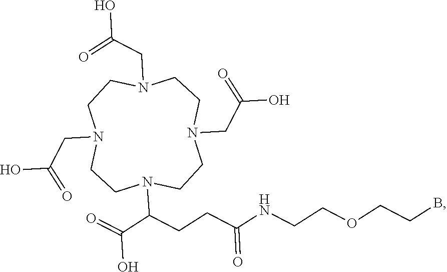

B being an IGF-1R antibody, an FGFR3 antibody, or a TEM-1 antibody, or an antigen-binding domain thereof;

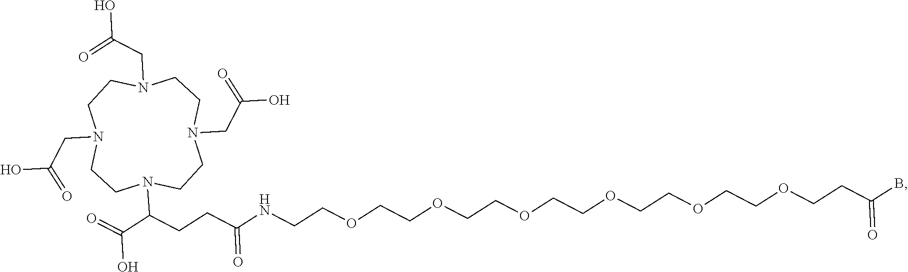

B being an IGF-1R antibody, an FGFR3 antibody, or a TEM-1 antibody, or an antigen-binding domain thereof;

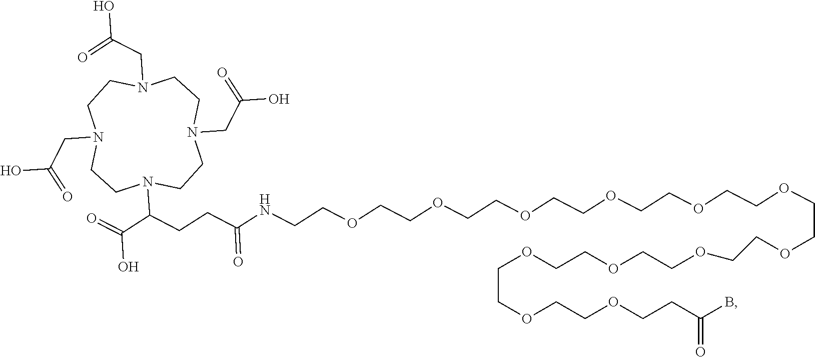

B being an IGF-1R antibody, an FGFR3 antibody, or a TEM-1 antibody, or an antigen-binding domain thereof,

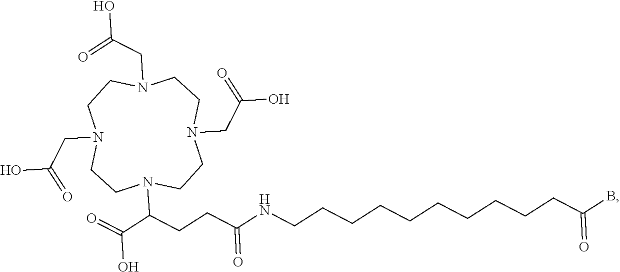

B being an IGF-1R antibody, an FGFR3 antibody, or a TEM-1 antibody, or an antigen-binding domain thereof.

In some embodiments, the combination therapy comprises a PD-1 inhibitor and an [²²⁵Ac]-radioimmunoconjugate that comprises ²²⁵Ac chelated with one of the following compounds:

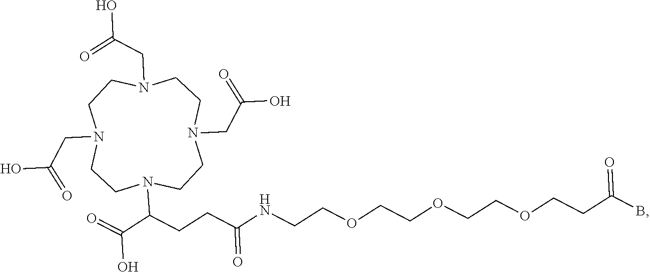

B being TAB-199 or AVE1642.

In some embodiments, the combination therapy comprises pembrolizumab and an [²²⁵Ac]-radioimmunoconjugate that comprises ²²⁵Ac chelated with the following compound:

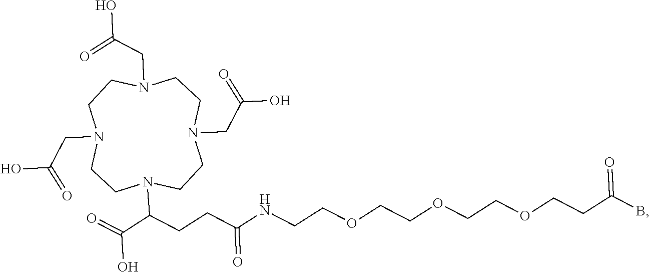

B being AVE1642.

### Subjects

In some disclosed methods, a therapy (e.g., comprising a therapeutic agent) is administered to a subject. In some embodiments, the subject is a mammal, e.g., a human.

In some embodiments, the subject has received or is receiving another therapy. For example, in some embodiments, the subject has received or is receiving a radioimmunoconjugate. In some embodiments, the subject has received or is receiving a checkpoint inhibitor.

In some embodiments, the subject has cancer or is at risk of developing cancer. For example, the subject may have been diagnosed with cancer. The cancer may be a primary cancer or a metastatic cancer. Subjects may have any stage of cancer, e.g., stage I, stage II, stage III, or stage IV with or without lymph node involvement and with or without metastases. Provided compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is a solid tumor.

In some embodiments, the solid tumor cancer is breast cancer (e.g., TNBC), non-small cell lung cancer, small cell lung cancer, pancreatic cancer, head and neck cancer, prostate cancer, colorectal cancer, cervical cancer, endometrial cancer, sarcoma, adrenocortical carcinoma, neuroendocrine cancer, Ewing's Sarcoma, multiple myeloma, or acute myeloid leukemia.

In some embodiments, the cancer is a non-solid (e.g., liquid (e.g., hematologic)) cancer.

### Administration and Dosage

### Effective and Lower Effective Doses

The present disclosure provides combination therapies in which the amounts of each therapeutic may or may not be, on their own, therapeutically effective. For example, provided are methods comprising administering a first therapy and a second therapy in amounts that together are effective to treat or ameliorate a disorder, e.g., cancer. In some embodiments, at least one of the first and second therapy is administered to the subject in a lower effective dose. In some embodiments, both the first and the second therapies are administered in lower effective doses.

In some embodiments, the first therapy comprises a radioimmunoconjugate and the second therapy comprises a checkpoint inhibitor.

In some embodiments, the first therapy comprises a checkpoint inhibitor and the second therapy comprises a radioimmunoconjugate.

In some embodiments, therapeutic combinations as disclosed herein are administered to a subject in a manner (e.g., dosing amount and timing) sufficient to cure or at least partially arrest the symptoms of the disorder and its complications. In the context of a single therapy (a “monotherapy”), an amount adequate to accomplish this purpose is defined as a “therapeutically effective amount,” an amount of a compound sufficient to substantially improve at least one symptom associated with the disease or a medical condition. The “therapeutically effective amount” typically varies depending on the therapeutic. For known therapeutic agents, the relevant therapeutically effective amounts may be known to or readily determined by those of skill in the art.

For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual. For example, a treatment may be therapeutically effective if it causes a cancer to regress or to slow the cancer's growth.

The dosage regimen (e.g., amounts of each therapeutic, relative timing of therapies, etc.) that is effective for these uses may depend on the severity of the disease or condition and the weight and general state of the subject. For example, the therapeutically effective amount of a particular composition comprising a therapeutic agent applied to mammals (e.g., humans) can be determined by the ordinarily skilled artisan with consideration of individual differences in age, weight, and the condition of the mammal. Because certain conjugates of the present disclosure exhibit an enhanced ability to target cancer cells and residualize, the dosage of these compounds can be lower than (e.g., less than or equal to about 90%, 75%, 50%, 40%, 30%, 20%, 15%, 12%1, 0%, 8%, 7%, 6%, 5%, 4%, 3%, 2%1, %, 0.5%, or 0.1% of) the equivalent dose of required for a therapeutic effect of the unconjugated agent. Therapeutically effective and/or optimal amounts can also be determined empirically by those of skill in the art. Thus, lower effective doses can also be determined by those of skill in the art.

To practice the methods of this invention, the [²²⁵Ac]-radioimmunoconjugate is typically administered at a dose of about 10 kBq to about 400 kBq/kg. In some embodiments, the [²²⁵Ac]-radioimmunoconjugate is administered at a dose of about 10 kBq to about 200 kBq/kg (e.g., about 10 kBq to about 150 kBq/kg, about 10 kBq to about 120 kBq/kg, about 10 kBq to about 100 kBq/kg; about 20 kBq to about 150 kBq/kg, about 20 kBq to about 120 kBq/kg, about 20 kBq to about 100 kBq/kg; about 30 kBq to about 150 kBq/kg, about 30 kBq to about 120 kBq/kg, about 30 kBq to about 100 kBq/kg; about 40 kBq to about 150 kBq/kg, about 40 kBq to about 120 kBq/kg, about 40 kBq to about 100 kBq/kg, or about 40 kBq to about 80 kBq/kg) of body weight of said patient.

In some embodiments, the [²²⁵Ac]-radioimmunoconjugate is administered at a dose of about 30 kBq to about 120 kBq/kg (e.g., about 35 kBq/kg, about 40 kBq/kg, about 45 kBq/kg, about 50 kBq/kg, about 55 kBq/kg, about 60 kBq/kg, about 65 kBq/kg, about 70 kBq/kg, about 75 kBq/kg, about 80 kBq/kg, about 85 kBq/kg, about 90 kBq/kg, about 95 kBq/kg, about 100 kBq/kg, about 105 kBq/kg, about 110 kBq/kg, or about 115 kBq/kg) of body weight of said patient.

In some embodiments, the [²²⁵Ac]-radioimmunoconjugate is administered as a unitary dosage of about 1-30 MBq (e.g., about 1-25 MBq, about 1-20 MBq, about 1-15 MBq, about 1-10 MBq; about 2-25 MBq, about 2-20 MBq, about 2-15 MBq, about 2-10 MBq; about 3-25 MBq, about 3-20 MBq, about 3-15 MBq, about 3-10 MBq; about 5-25 MBq, about 5-20 MBq, about 5-15 MBq, about 5-10 MBq) to said patient.

In some embodiments, the [²²⁵Ac]-radioimmunoconjugate is administered as a unitary dosage of about 5-15 MBq (e.g., about 6 MBq, about 7 MBq, about 8 MBq, about 9 MBq, about 10 MBq, about 11 MBq, about 12 MBq, about 13 MBq, or about 14 MBq) to said patient.

In some embodiments, the [²²⁵Ac]-radioimmunoconjugate is administered as a unitary dosage of about 20-30 MBq (e.g., about 21 MBq, about 22 MBq, about 23 MBq, about 24 MBq, about 25 MBq, about 26 MBq, about 27 MBq, about 28 MBq, or about 29 MBq) to said patient.

Single or multiple administrations of a composition (e.g., a pharmaceutical composition comprising a therapeutic agent) can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

In some embodiments, the above-described unitary dosage can be administered to a subject (e.g., a patient) twice daily, three times daily, or four times daily. For example, when the [²²⁵Ac]-radioimmunoconjugate is administered as a unitary dosage of about 10-30 MBq, it can be administered to a patient twice daily in a total dosage of about 20 MBq to about 60 MBq daily.

In the disclosed combination therapy methods, the first and second therapies may be administered sequentially or concurrently to a subject. For example, a first composition comprising a first therapeutic agent and a second composition comprising a second therapeutic agent may be administered sequentially or concurrently to a subject. Alternatively, or additionally, a composition comprising a combination of a first therapeutic agent and a second therapeutic agent may be administered to the subject.

In some embodiments, the radioimmunoconjugate is administered in a single dose. In some embodiments, the radioimmunoconjugate is administered more than once. When the radioimmunoconjugate is administered more than once, the dose of each administration may be the same or different.

In some embodiments, the checkpoint inhibitor is administered in a single dose. In some embodiments, the checkpoint inhibitor is administered more than once, e.g., at least twice, at least three times, etc. In some embodiments, the checkpoint inhibitor is administered multiple times according to a regular or semi-regular schedule, e.g., once every approximately two weeks, once a week, twice a week, three times a week, or more than three times a week. When the checkpoint inhibitor is administered more than once, the dose of each administration may be the same or different. For example, the checkpoint inhibitor may be administered in an initial dose amount, and then subsequent dosages of the checkpoint inhibitor may be higher or lower than the initial dose amount.

In some embodiments, the first dose of the checkpoint inhibitor is administered at the same time as the first dose of the radioimmunoconjugate. In some embodiments, the first dose of the checkpoint inhibitor is administered before the first dose of radioimmunoconjugate. In some embodiments, the first dose of the checkpoint inhibitor is administered after the first dose of radioimmunoconjugate. In some embodiments, subsequent doses of the checkpoint inhibitor are administered.

In some embodiments, radioimmunoconjugates (or a composition thereof) and checkpoint inhibitors (or a composition thereof) are administered within 28 days (e.g., within 14, 7, 6, 5, 4, 3, 2, or 1 day(s)) of each other.

In some embodiments, radioimmunoconjugates (or a composition thereof) and checkpoint inhibitors (or a composition thereof) are administered within 90 days (e.g., within 80, 70, 60, 50, 40, 30, 20, 10, 5, 4, 3, 2, or 1 day(s)) of each other. In various embodiments the checkpoint inhibitor is administered at the same time as radioimmunoconjugate. In various embodiments, the checkpoint inhibitor is administered multiple times after the first administration of radioimmunoconjugate.

In some embodiments, compositions (such as compositions comprising radioimmunoconjugates) are administered for radiation treatment planning or diagnostic purposes. When administered for radiation treatment planning or diagnostic purposes, compositions may be administered to a subject in a diagnostically effective dose and/or an amount effective to determine the therapeutically effective dose. In some embodiments, a first dose of disclosed conjugate or a composition (e.g., pharmaceutical composition) thereof is administered in an amount effective for radiation treatment planning, followed administration of a combination therapy including a conjugate as disclosed herein and another therapeutic.

Pharmaceutical compositions comprising one or more agents (e.g., radioimmunoconjugates and/or checkpoint inhibitors) can be formulated for use in accordance with disclosed methods and systems in a variety of drug delivery systems. One or more physiologically acceptable excipients or carriers can also be included in the composition for proper formulation. Examples of suitable formulations are found in Remington's Pharmaceutical Sciences, Mack Publishing Company, Philadelphia, PA, 17th ed., 1985. For a brief review of methods for drug delivery, see, e.g., Langer (Science 249:1527-1533, 1990).

### Formulations

Pharmaceutical compositions may be formulated for parenteral, intranasal, topical, oral, or local administration, such as by a transdermal means, for prophylactic and/or therapeutic treatment. Pharmaceutical compositions can be administered parenterally (e.g., by intravenous, intramuscular, or subcutaneous injection), or by oral ingestion, or by topical application or intraarticular injection at areas affected by the vascular or cancer condition. Examples of additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Also specifically contemplated are sustained release administration, by such means as depot injections or erodible implants or components. Suitable compositions include compositions comprising include agents (e.g., compounds as disclosed herein) dissolved or suspended in an acceptable carrier, preferably an aqueous carrier, e.g., water, buffered water, saline, or PBS, among others, e.g., for parenteral administration. Compositions may contain pharmaceutically acceptable auxiliary substances to approximate physiological conditions, such as pH adjusting and buffering agents, tonicity adjusting agents, wetting agents, or detergents, among others. In some embodiments, compositions are formulated for oral delivery; for example, compositions may contain inert ingredients such as binders or fillers for the formulation of a unit dosage form, such as a tablet or a capsule. In some embodiments, compositions are formulated for local administration; for example, compositions may contain inert ingredients such as solvents or emulsifiers for the formulation of a cream, an ointment, a gel, a paste, or an eye drop.

Compositions may be sterilized, e.g., by conventional sterilization techniques, or sterile filtered. Aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 6 and 7, such as 6 to 6.5. In some embodiments, compositions in solid form are packaged in multiple single dose units, each containing a fixed amount of the above-mentioned agent or agents, such as in a sealed package of tablets or capsules. In some embodiments, compositions in solid form are packaged in a container for a flexible quantity, such as in a squeezable tube designed for a topically applicable cream or ointment.

In some embodiments, provided are compositions comprising an [²²⁵Ac]-radioimmunoconjugate as described herein in an amount that represents a lower effective dose.

### Kits

In some embodiments, provided are kits that comprise (1) a composition comprising an [²²⁵Ac]-radioimmunoconjugate as described herein and (2) instructions for administering the composition in combination with a checkpoint inhibitor.

In some embodiments, provided are kits that comprise (1) a composition comprising a checkpoint inhibitor and (2) instructions for administering the composition in combination with a [²²⁵Ac]-radioimmunoconjugate as described herein.

### Effects

In some embodiments, methods of the present disclosure result in a therapeutic effect. In some embodiments, the therapeutic effect comprises an immune response, for example, an immune response comprises an increase in T cells, e.g. CD8+ (e.g., IFNγ-producing CD8+ cells) and/or CD4+ cells. In some embodiments, the T cells comprise T cells specific for a tumor-associated antigen or tumor-specific antigen expressed on the cancer being treated or ameliorated. In some embodiments, the increase in T cells is observed in the tumor relative to the spleen.

In some embodiments, the step of administering results in at least 15%, at least 20%, at least 25%, at least 30%, at least 35%, at least 40%, at least 45%, at least 50%, at least 55%, at least 60%, at least 65%, or at least 70% of the total T cell population in a sample in the mammal being specific for the tumor-associated antigen or tumor-specific antigen. In some embodiments, the sample is a tumor sample.

In some embodiments, the therapeutic effect comprises a decrease in tumor volume (e.g., at least partial tumor regression), a stable tumor volume, or a reduced rate of increase in tumor volume. In some embodiments, the therapeutic effect comprises a decreased incidence of recurrence or metastasis.

In some embodiments, the therapeutic effect comprises tumor regression, that is, a reduction in tumor volume. In some embodiments, the tumor regression is characterized by a decrease in tumor volume of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the tumor volume before treatment initiation. In some embodiments, the therapeutic effect comprises complete tumor regression.

In some embodiments, the tumor regression (whether partial or complete) is durable in that the tumor volume does not increase substantially again after decreasing over a period of time. In some embodiments, the tumor regression is durable over a period of at least five days, at least ten days, at least 15 days, at least 20 days, at least 23 days, at least 25 days, at least 26 days, at least 27 days, at least 28 days, at least 29 days, or at least 30 days after initiation of treatment.

### Other Agents

In some embodiments, disclosed methods further include administering an antiproliferative agent, radiation sensitizer, or an immunoregulatory or immunomodulatory agent.

By “antiproliferative” or “antiproliferative agent,” as used interchangeably herein, is meant any anticancer agent, including those antiproliferative agents listed in Table 2, any of which can be used in combination with a radioimmunoconjugate to treat a condition or disorder. Antiproliferative agents also include organo-platinum derivatives, naphtoquinone and benzoquinone derivatives, chrysophanic acid and anthroquinone derivatives thereof.

By “immunoregulatory agent” or “immunomodulatory agent,” as used interchangeably herein, is meant any immuno-modulator, including those listed in Table 2, any of which can be used in combination with a radioimmunoconjugate.

As used herein, “radiation sensitizer” includes any agent that increases the sensitivity of cancer cells to radiation therapy. Radiation sensitizers may include, but are not limited to, 5-fluorouracil, analogs of platinum (e.g., cisplatin, carboplatin, oxaliplatin), gemcitabine, EGFR antagonists (e.g., cetuximab, gefitinib), farmesyltransferase inhibitors, COX-2 inhibitors, bFGF antagonists, and VEGF antagonists.

TABLE 2

| | | |
|---|---|---|
| Alkylating agents | Busulfan | Chlorambucil |
| | dacarbazine | procarbazine |
| | ifosfamide | altretamine |
| | hexamethylmelamine | estramustine phosphate |
| | thiotepa | mechlorethamine |
| | dacarbazine | streptozocin |
| | lomustine | temozolomide |
| | cyclophosphamide | Semustine |
| Platinum agents | spiroplatin | lobaplatin (Aeterna) |
| | tetraplatin | satraplatin (Johnson |
| | ormaplatin | Matthey) |
| | iproplatin | BBR-3464 (Hoffmann-La |
| | picoplatin | Roche) |
| | oxaliplatin | Miriplatin |
| | carboplatin | AP-5280 (Access) |
| | | cisplatin |
| Antimetabolites | azacytidine | trimetrexate |
| | Floxuridine | deoxycoformycin |
| | 2-chlorodeoxyadenosine | pentostatin |
| | 6-mercaptopurine | hydroxyurea |
| | 6-thioguanine | decitabine (SuperGen) |
| | cytarabine | clofarabine (Bioenvision) |
| | 2-fluorodeoxy cytidine | irofulven (MGI Pharma) |
| | methotrexate | DMDC (Hoffmann-La |
| | tomudex | Roche) |
| | fludarabine | ethynylcytidine (Taiho) |
| | raltitrexed | gemcitabine |
| | | capecitabine |
| Topoisomerase | amsacrine | exatecan mesylate |
| inhibitors | epirubicin | (Daiichi) |
| | etoposide | quinamed (ChemGenex) |
| | teniposide or mitoxantrone | gimatecan (Sigma-Tau) |
| | 7-ethyl-10-hydroxy- | diflomotecan (Beaufour- |
| | camptothecin | Ipsen) |
| | dexrazoxanet (TopoTarget) | TAS-103 (Taiho) |
| | pixantrone (Novuspharma) | elsamitrucin (Spectrum) |
| | rebeccamycin analogue | Edotecarin |
| | (Exelixis) | Cositecan |
| | BBR-3576 (Novuspharma) | Belotecan |
| | rubitecan (SuperGen) | hydroxycamptothecin (SN- |
| | irinotecan (CPT-11) | 38) |
| | topotecan | |
| Antitumor | valrubicin | azonafide |
| antibiotics | therarubicin | anthrapyrazole |
| | idarubicin | oxantrazole |
| | rubidazone | losoxantrone |
| | plicamycin | Sabarubicin |
| | porfiromycin | Epirubicin |
| | mitoxantrone (novantrone) | mitoxantrone |
| | amonafide | doxorubicin |
| Antimitotic | colchicine | E7010 (Abbott) |
| agents | vinblastine | PG-TXL (Cell |
| | vindesine | Therapeutics) |
| | dolastatin 10 (NCI) | IDN 5109 (Bayer) |
| | rhizoxin (Fujisawa) | A 105972 (Abbott) |
| | mivobulin (Warner- | A 204197 (Abbott) |
| | Lambert) | LU 223651 (BASF) |
| | cemadotin (BASF) | D 24851 (ASTAMedica) |
| | RPR 109881A (Aventis) | ER-86526 (Eisai) |
| | TXD 258 (Aventis) | combretastatin A4 (BMS) |
| | epothilone B (Novartis) | isohomohalichondrin-B |
| | T 900607 (Tularik) | (PharmaMar) |
| | T 138067 (Tularik) | ZD 6126 (AstraZeneca) |
| | cryptophycin 52 (Eli Lilly) | AZ10992 (Asahi) |
| | vinflunine (Fabre) | IDN-5109 (Indena) |
| | auristatin PE (Teikoku | AVLB (Prescient |
| | Hormone) | NeuroPharma) |
| | BMS 247550 (BMS) | azaepothilone B (BMS) |
| | BMS 184476 (BMS) | BNP-7787 (BioNumerik) |
| | BMS 188797 (BMS) | CA-4 prodrug (OXiGENE) |
| | taxoprexin (Protarga) | dolastatin-10 (NIH) |
| | SB 408075 | CA-4 (OXiGENE) |
| | (GlaxoSmithKline) | docetaxel |
| | Vinorelbine | vincristine |
| | Trichostatin A | paclitaxel |
| Aromatase | aminoglutethimide | YM-511 (Yamanouchi) |
| inhibitors | atamestane (BioMedicines) | formestane |
| | letrozole | exemestane |
| | anastrazole | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | edotreotide (Novartis) |
| | glufosfamide (Baxter | mafosfamide (Baxter |
| | International) | International) |
| | albumin + 32P (Isotope | apaziquone (Spectrum |
| | Solutions) | Pharmaceuticals) |
| | thymectacin (NewBiotics) | O6 benzyl guanine |
| | | (Paligent) |
| Farnesyltransferase | arglabin (NuOncology | tipifarnib (Johnson & |
| inhibitors | Labs) | Johnson) |
| | lonafarnib (Schering- | perillyl alcohol (DOR |
| | Plough) | BioPharma) |
| | BAY-43-9006 (Bayer) | |
| Pump inhibitors | CBT-1 (CBA Pharma) | zosuquidar |
| | tariquidar (Xenova) | trihydrochloride (Eli Lilly) |
| | MS-209 (Schering AG) | biricodar dicitrate (Vertex) |
| Histone | tacedinaline (Pfizer) | pivaloyloxymethyl |
| acetyltransferase | SAHA (Aton Pharma) | butyrate (Titan) |
| inhibitors | MS-275 (Schering AG) | depsipeptide (Fujisawa) |
| Metalloproteinase | Neovastat (Aeterna | CMT-3 (CollaGenex) |
| inhibitors | Laboratories) | BMS-275291 (Celltech) |
| | marimastat (British | |
| | Biotech) | |
| Ribonucleoside | gallium maltolate (Titan) | tezacitabine (Aventis) |
| reductase inhibitors | triapine (Vion) | didox (Molecules for |
| | | Health) |
| TNF alpha | virulizin (Lorus | revimid (Celgene) |
| agonists/antagonists | Therapeutics) | |
| | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & | alitretinoin (Ligand) |
| receptor agonists | Johnson) | |
| | LGD-1550 (Ligand) | |
| Immuno- | interferon | dexosome therapy |
| modulators | oncophage (Antigenics) | (Anosys) |
| | GMK (Progenics) | pentrix (Australian Cancer |
| | adenocarcinoma vaccine | Technology) |
| | (Biomira) | ISF-154 (Tragen) |
| | CTP-37 (AVI BioPharma) | cancer vaccine (Intercell) |
| | IRX-2 (Immuno-Rx) | norelin (Biostar) |
| | PEP-005 (Peplin Biotech) | BLP-25 (Biomira) |
| | synchrovax vaccines (CTL | MGV (Progenics) |
| | Immuno) | β-alethine (Dovetail) |
| | melanoma vaccine (CTL | CLL therapy (Vasogen) |
| | Immuno) | Ipilimumab (BMS), |
| | p21 RAS vaccine | CM-10 (cCam |
| | (GemVax) | Biotherapeutics) |
| | MAGE-A3 (GSK) | atezolizumab (Genentech) |
| | nivolumab (BMS) | |
| | abatacept (BMS) | |
| | pembrolizumab (Merck) | |
| Hormonal and | estrogens | dexamethasone |
| antihormonal | conjugated estrogens | prednisone |
| agents | ethinyl estradiol | methylprednisolone |
| | chlortrianisen | prednisolone |
| | idenestrol | aminoglutethimide |
| | hydroxyprogesterone | leuprolide |
| | caproate | octreotide |
| | medroxyprogesterone | mitotane |
| | testosterone | P-04 (Novogen) |
| | testosterone propionate; | 2-methoxyestradiol |
| | fluoxymesterone | (EntreMed) |
| | methyltestosterone | arzoxifene (Eli Lilly) |
| | diethylstilbestrol | tamoxifen |
| | megestrol | toremofine |
| | bicalutamide | goserelin |
| | flutamide | Leuporelin |
| | nilutamide | bicalutamide |
| Photodynamic | talaporfin (Light Sciences) | Pd-bacteriopheophorbide |
| agents | Theralux | (Yeda) |
| | (Theratechnologies) | Motexafin lutetium |
| | motexafin gadolinium | hypericin |
| | (Pharmacyclics) | |
| Kinase Inhibitors | imatinib (Novartis) | EKB-569 (Wyeth) |
| | leflunomide | kahalide F (PharmaMar) |
| | (Sugen/Pharmacia) | CEP-701 (Cephalon) |
| | ZD1839 (AstraZeneca) | CEP-751 (Cephalon) |
| | erlotinib (Oncogene | MLN518 (Millenium) |
| | Science) | PKC412 (Novartis) |
| | canertinib (Pfizer) | Phenoxodiol (Novogen) |
| | squalamine (Genaera) | C225 (ImClone) |
| | SU5416 (Pharmacia) | rhu-Mab (Genentech) |
| | SU6668 (Pharmacia) | MDX-H210 (Medarex) |
| | ZD4190 (AstraZeneca) | 2C4 (Genentech) |
| | ZD6474 (AstraZeneca) | MDX-447 (Medarex) |
| | vatalanib (Novartis) | ABX-EGF (Abgenix) |
| | PKI166 (Novartis) | IMC-1C11 (ImClone) |
| | GW2016 | Tyrphostins |
| | (GlaxoSmithKline) | Gefitinib (Iressa) |
| | EKB-509 (Wyeth) | PTK787 (Novartis) |
| | trastuzumab (Genentech) | EMD 72000 (Merck) |
| | OSI-774 (Tarceva ™) | Emodin |
| | CI-1033 (Pfizer) | Radicinol |
| | SU11248 (Pharmacia) | Vemurafenib (B-Raf |
| | RH3 (York Medical) | enzyme inhibitor, Daiichi |
| | Genistein | Sankyo) |
| | Radicinol | |
| | Met-MAb (Roche) | |

| | |
|---|---|
| SR-27897 (CCK A inhibitor, Sanofi-Synthelabo) | ceflatonin (apoptosis promotor, ChemGenex) |
| tocladesine (cyclic AMP agonist, Ribapharm) | BCX-1777 (PNP inhibitor, BioCryst) |
| alvocidib (CDK inhibitor, Aventis) | ranpirnase (ribonuclease stimulant, Alfacell) |
| CV-247 (COX-2 inhibitor, Ivy Medical) | galarubicin (RNA synthesis inhibitor, Dong- |
| P54 (COX-2 inhibitor, Phytopharm) | A) |
| CapCell ™ (CYP450 stimulant, Bavarian Nordic) | tirapazamine (reducing agent, SRI |
| GCS-100 (gal3 antagonist, GlycoGenesys) | International) |
| G17DT immunogen (gastrin inhibitor, Aphton) | N-acetylcysteine (reducing agent, Zambon) |
| efaproxiral (oxygenator, Allos Therapeutics) | R-flurbiprofen (NF-kappaB inhibitor, Encore) |
| PI-88 (heparanase inhibitor, Progen) | 3CPA (NF-kappaB inhibitor, Active Biotech) |
| tesmilifene (histamine antagonist, YM | seocalcitol (vitamin D receptor agonist, Leo) |
| BioSciences) | 131-I-TM-601 (DNA antagonist, |
| histamine (histamine H2 receptor agonist, Maxim) | TransMolecular) |
| tiazofurin (IMPDH inhibitor, Ribapharm) | eflornithine (ODC inhibitor, ILEX Oncology) |
| cilengitide (integrin antagonist, Merck KGaA) | minodronic acid (osteoclast inhibitor, |
| SR-31747 (IL-1 antagonist, Sanofi-Synthelabo) | Yamanouchi) |
| CCI-779 (mTOR kinase inhibitor, Wyeth) | indisulam (p53 stimulant, Eisai) |
| exisulind (PDE V inhibitor, Cell Pathways) | aplidine (PPT inhibitor, PharmaMar) |
| CP-461 (PDE V inhibitor, Cell Pathways) | gemtuzumab (CD33 antibody, Wyeth Ayerst) |
| AG-2037 (GARFT inhibitor, Pfizer) | PG2 (hematopoiesis enhancer, |
| WX-UK1 (plasminogen activator inhibitor, Wilex) | Pharmagenesis) |
| PBI-1402 (PMN stimulant, ProMetic | Immunol ™ (triclosan oral rinse, Endo) |
| LifeSciences) | triacetyluridine (uridine prodrug, Wellstat) |
| bortezomib (proteasome inhibitor, Millennium) | SN-4071 (sarcoma agent, Signature |
| SRL-172 (T cell stimulant, SR Pharma) | BioScience) |
| TLK-286 (glutathione S transferase inhibitor, | TransMID-107 ™ (immunotoxin, KS |
| Telik) | Biomedix) |
| PT-100 (growth factor agonist, Point Therapeutics) | PCK-3145 (apoptosis promotor, Procyon) |
| midostaurin (PKC inhibitor, Novartis) | doranidazole (apoptosis promotor, Pola) |
| bryostatin-1 (PKC stimulant, GPC Biotech) | CHS-828 (cytotoxic agent, Leo) |
| CDA-II (apoptosis promotor, Everlife) | trans-retinoic acid (differentiator, NIH) |
| SDX-101 (apoptosis promotor, Salmedix) | MX6 (apoptosis promotor, MAXIA) |
| rituximab (CD20 antibody, Genentech | apomine (apoptosis promotor, ILEX |
| carmustine | Oncology) |
| Mitoxantrone | urocidin (apoptosis promotor, Bioniche) |
| Bleomycin | Ro-31-7453 (apoptosis promotor, La Roche) |
| Absinthin | brostallicin (apoptosis promotor, Pharmacia) |
| Chrysophanic acid | β-lapachone |
| Cesium oxides | gelonin |
| BRAF inhibitors, | cafestol |
| PD-L1 inhibitors | kahweol |
| MEK inhibitors | caffeic acid |
| bevacizumab | Tyrphostin AG |
| angiogenesis inhibitors | PD-1 inhibitors |
| dabrafenib | CTLA-4 inhibitors |
| | sorafenib |

### EXAMPLES

### Example 1. Single Agent Efficacy of Checkpoint Inhibitors in the CT-26 Syngeneic Model was Observed

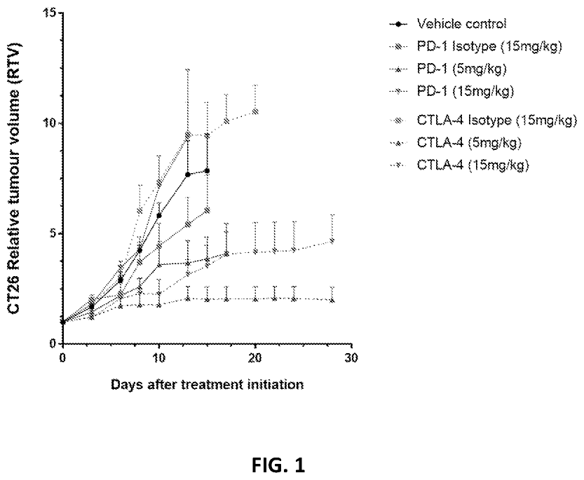

A single agent efficacy study of two checkpoint inhibitors (PD-1 and CTLA-4) was conducted in the CT-26 model, a murine colon carcinoma model. It is known that these carcinomas are partially sensitive to α-PD-1 mAbs and sensitive to α-CTLA-4 mAbs. Mice were injected i.p. with either 5 or 15 mg/kg i.p. of either the α-PD-1 mAb or the α-CTLA-4 mAb. The α-PD-1 mAb group was dosed twice a week for four weeks. The α-CTLA-4 mAb group was dosed only 3 times a day, 3 days apart. CTLA-4 treatment was more efficacious than PD-1 treatment, as expected for this model. In both treatment groups, 5 mg/kg appeared to be the most efficacious dose for impairing tumor growth. See FIG. 1. CD8+/CD4+ T cell recruitment following the different treatments is also measured using immunohistochemistry and flow cytometry techniques.

### Example 2. Selection of hIGF-1R Expression CT26 Clones to Generate Mouse Cancer Models

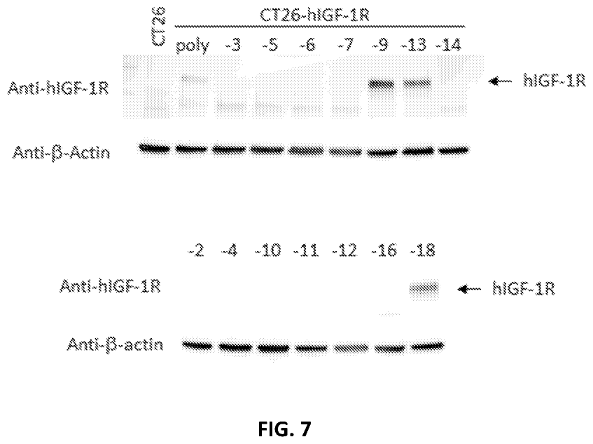

CT26 cells were stably transfected with human IGF-1R plasmid. Western blot analysis was conducted for the presence of hIGF-1R for the selection of hIGF-1R expressing clones. See FIG. 7. The best clones were chosen based on both in vitro and in vivo characteristics. The resulting cell lines were xenografted into mice to generate mouse models for testing [²²⁵Ac]-Compound D (TAB-199 conjugated with Compound A1 and radiolabeled with [²²⁵Ac]; see Example 5-B) and/or other radioimmunoconjugates (e.g., conjugates comprising AVE1642) and additional synergies with immune checkpoint inhibitors, as described further below.

### Example 3. [¹⁷⁷Lu]-Compound B Biodistribution in the CT-26 Syngeneic Model

MAB391, a murine monoclonal antibody against IGF-1R (see, e.g., F. J. Calzone et al., PLoS One. 2013; 8(2): e55135), was conjugated with Compound A1 (a bifunctional chelate represented by the structure shown below) and radiolabeled with Lu-177 using methods well known in the art to form [¹⁷⁷Lu]-Compound B.

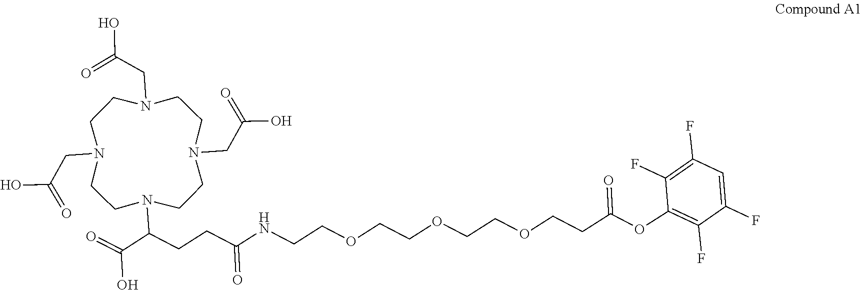 [SMILES: O=C(O)CN1CCN(CC(=O)O)CCN(C(CCC(=O)NCCOCCOCCOCCC(=O)Oc2c(F)c(F)cc(F)c2F)C(=O)O)CCN(CC(=O)O)CC1]

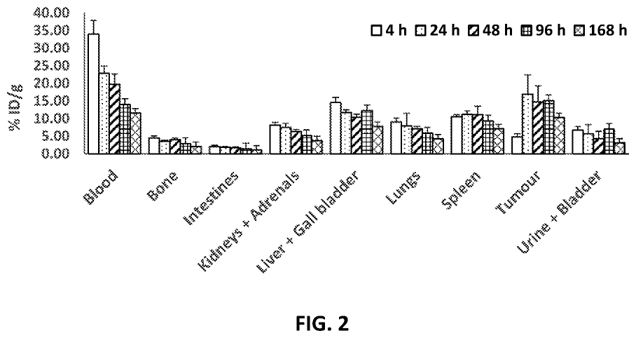

The ability of [¹⁷⁷Lu]-Compound B to target antigen expressing mouse IGF-1R overexpressing tumors in vivo was demonstrated using the CT-26 syngeneic model. Tumor uptake was steady at 15-17% injected dose/g (ID/g) from 24-96 hours post injection. See FIG. 2.

### Example 4. Enhanced Efficacy of [²²⁵Ac]-Compound C in Immunocompetent Vs. In Immunodeficient Mice

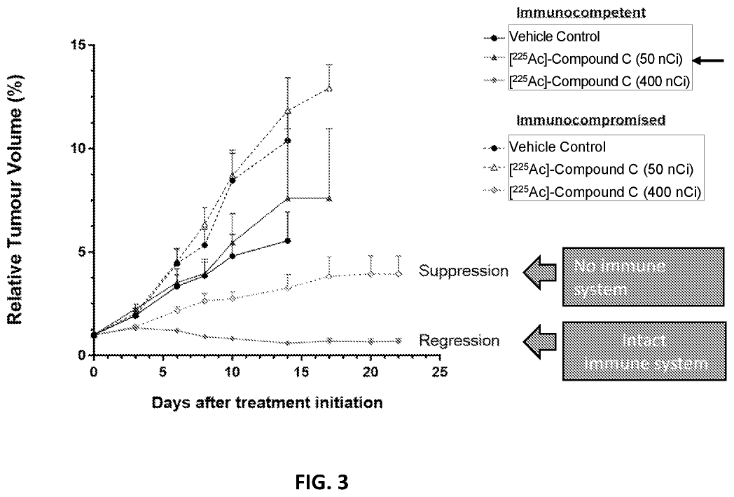

MAB391, a murine monoclonal antibody against IGF-1R, was conjugated with Compound A1 and radiolabeled with [²²⁵Ac] using standard techniques to form [²²⁵Ac]-Compound C. An efficacy study of [²²⁵Ac]-Compound C in immunocompetent and in immunodeficient mice was conducted using a 92.5 kBq/kg or 740 kBq/kg (50 nCi or 400 nCi) dose of [²²⁵Ac]-Compound C. It was found that [²²⁵Ac]-Compound C had enhanced efficacy in reducing tumor volume in mice with an intact immune system relative to mice with no immune system. See FIG. 3.

Of note, the dosage of 50 nCi in mice corresponds to 92.5 kBq/kg, which is equivalent to about 7 MBq in human (human equivalent dose); and the dosage of 400 nCi in mice corresponds to 740 kBq/kg, which is equivalent to about 55 MBq in human (human equivalent dose).

### Example 5-A. Synergy Between [²²⁵Ac]-Compound C and α-CTLA-4/PD-1 Treatment in the CT26 Syngeneic Mouse Model

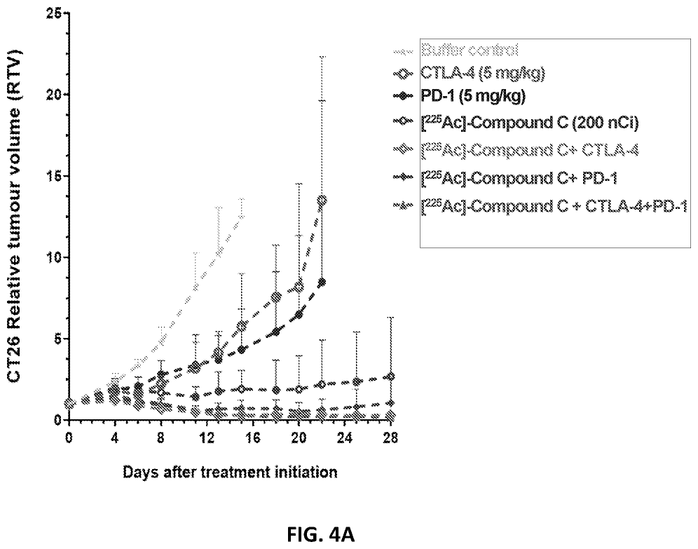

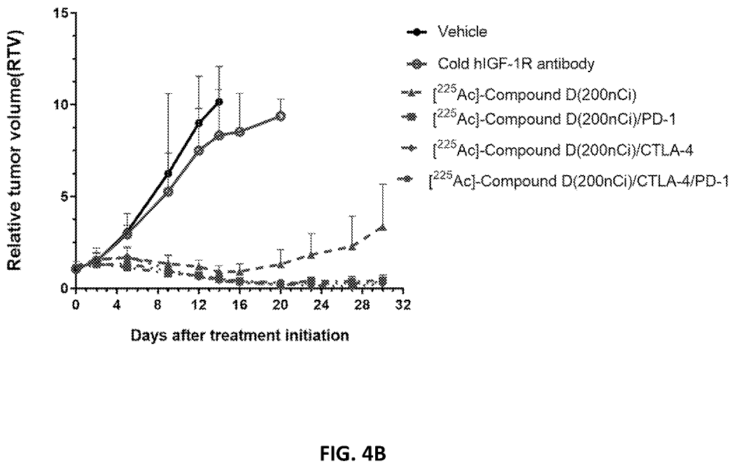

An in vivo synergy study was conducted to test the effect of [²²⁵Ac]-Compound C (as described in Example 4) and checkpoint inhibitors, α-CTLA-4 and α-PD-1 antibodies, on relative tumor volume in the CT26 mouse model. Mice treated either with the CTLA-4 inhibitor alone or the PD-1 inhibitor alone showed modest reductions in relative tumor volume when compared to the vehicle control groups. Mice treated with [²²⁵Ac]-Compound C at a 370 kBq/kg (or 200 nCi) dose demonstrated greater reductions in tumor volume relative to the vehicle control group or the groups administered CTLA-4 inhibitor or PD-1 inhibitor alone. However, when [²²⁵Ac]-Compound C was co-administered at a 370 kBq/kg dose with ether CTLA-4 or PD-1 or both, a synergistic effect was seen—co-administration resulted in significantly smaller tumor volume when compared to treatment with [²²⁵Ac]-Compound C, or when compared to treatment with the CTLA-4 inhibitor or the PD-1 inhibitor alone. See FIG. 4A.

Of note, the dosage of 200 nCi in mice corresponds to 370 kBq/kg, which is equivalent to about 28 MBq in human (human equivalent dose).

### Example 5-B. Synergy Between [²²⁵Ac]-Compound D and α-CTLA-4/PD-1 Treatment in the CT26 Syngeneic Mouse Model

TAB-199, a human monoclonal antibody against IGF-1R (see, e.g., https://www.antibodypedia.com/gene/4140/IGF1R/antibody/2726933/TAB-199), was conjugated with Compound A1 and radiolabeled with [²²⁵Ac] using standard techniques known in the field to form [²²⁵Ac]-Compound D.

An in vivo synergy study was conducted to test the effect of [²²⁵Ac]-Compound D and checkpoint inhibitors, α-CTLA-4 and α-PD-1 antibodies, on relative tumor volume in the CT26 mouse model. Mice treated with [²²⁵Ac]-Compound D at the dose of 370 kBq/kg (or 200 nCi) demonstrated only transient tumor regression, followed by tumor regrowth. However, when [²²⁵Ac]-Compound D was co-administered at 370 kBq/kg with ether CTLA-4 or PD-1 or both, a synergistic effect was observed to show durable tumor regression—co-administration resulted in significantly smaller tumor volume when compared to treatment with [²²⁵Ac]-Compound D. See FIG. 4B.

Of note, the dosage of 200 nCi in mice corresponds to 370 kBq/kg, which is equivalent to about 28 MBq in human (human equivalent dose).

Blood samples were collected from mice treated with 200 nCi [²²⁵Ac]-Compound D with or without checkpoint inhibitors 2 days before and 12 days after treatment initiation and analysis of T cell receptor repertoire was conducted using the ImmunoSEQ technology (See, e.g., Wolf K, DiPaolo D., Immunosequencing: accelerating discovery in immunology and medicine. Curr Trends Immunol 2016; 17:85-93; Liu X, Wu J. History, applications, and challenges of immune repertoire research. Cell Biol Toxicol 2018; 34(6):441-57). The results suggested that treatment with [²²⁵Ac]-Compound D induced a more clonal T cell response which suggests immunoconjugate-elicited immune activation.

### Example 6. Development of Protective Immunity in [²²⁵Ac]-Compound C Retreated Mice Upon CT26 Re-Challenge

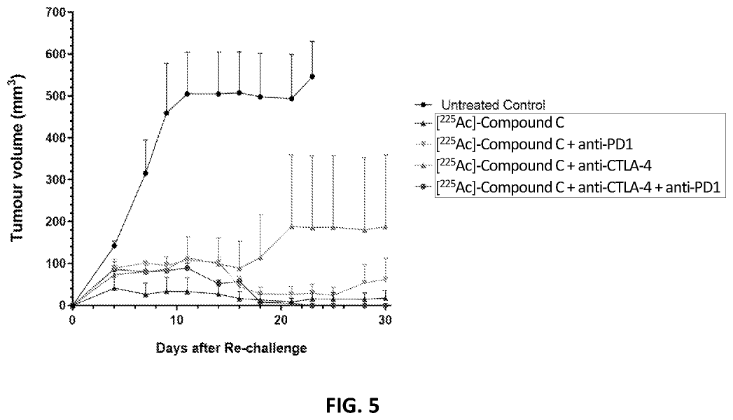

A re-challenge experiment was conducted to test the development of protective immunity in [²²⁵Ac]-Compound C treated mice upon CT26 re-challenge. Mice had been previously treated with either [²²⁵Ac]-Compound C alone or in combination with an α-CTLA-4 or α-PD-1 antibody. Naïve mice were used as controls. All mice previously treated with [²²⁵Ac]-Compound C+/− an anti-CTLA-4 or anti-PD-1 antibody were protected from tumor challenge, suggesting development of protective T cell immunity. See FIG. 5.

### Example 7. Cytokine Response and T-Cell Recruitment after [²²⁵Ac]-Compound C Treatment

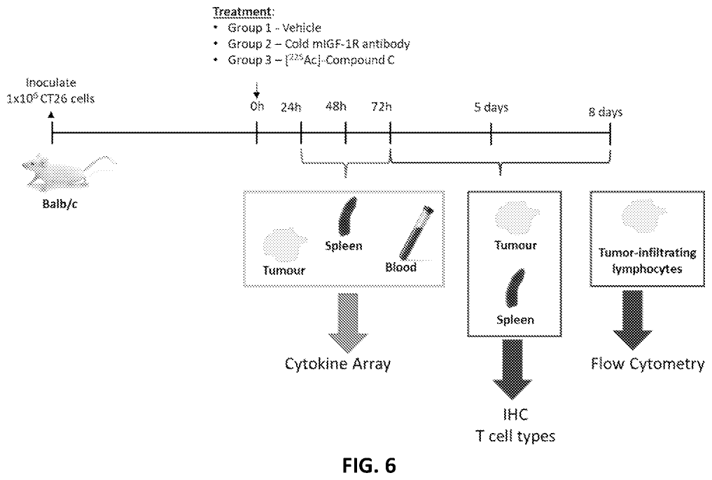

Cytokine response and T-cell recruitment after [²²⁵Ac]-Compound C treatment were measured. Mice were inoculated with 1×10⁶ CT26 cells. Mice were then treated with either [²²⁵Ac]-Compound C, the unconjugated MAB391 antibody or vehicle. Samples from the tumor, spleen and blood plasma were analyzed for the presence of cytokines at 24, 48, or 72 hours. Additional samples were taken from the tumor and spleen at 72 hours, 5 days and 8 days for immunohistochemistry to assess the presence of different T-cell types. Finally, at 8 days, tumor-infiltrating lymphocytes were extracted, isolated and quantified using flow cytometry. See FIG. 6.

Changes in cytokine expression were seen in tumors treated with [²²⁵Ac]-Compound C in comparison to the unconjugated MAB391 antibody, as shown in Table 3 below:

TABLE 3
Cytokine expression changes

| Tumor | Fold change | |
|---|---|---|
| Cytokine | ([²²⁵Ac]- | |
| (72 h) | Compound C: MAB391) | Function |
| IL-1Ra | 2.0 | Inhibits the pro-inflammatory |
| | | effect of IL-1b |
| CXCL10 | 2.7 | Chemoattraction of T cells, |
| | | NK cells, monocytes/macrophages, |
| | | and dendritic cells |
| CXCL11 | 1.9 | Chemoattraction of activated T |
| | | cells |
| TIMP-1 | 1.8 | Inhibitor of MMPs |
| MCP-1 | 0.2 | Chemoattraction of |
| | | monocytes/macrophages |
| CCL12 | 0.2 | Chemoattraction of eosinophils, |
| | | monocytes, and lymphocytes |
| CCL3 | 0.4 | Recruitment and activation of |
| | | leukocytes |

### Example 8. Combination Therapies Result in Increased Tumor-Associated Antigen-Specific CD8+ T Cells in Both the Spleen and Tumor Itself

[²²⁵Ac]-Compound D (see Example 5-B) is a radioimmunoconjugate comprising human monoclonal IGF-1R antibody TAB-199 labeled with Actinium-225 (²²⁵Ac). Combinations with [²²⁵Ac]-Compound D and checkpoint inhibitors (α-PD-1, α-CTLA-4, or both α-PD-1 and α-CTLA-4) were tested in the CT26 syngeneic mouse model. Mice were re-challenged with CT26 cells at day 28 after initial tumor inoculation.

CD8+ and CD4+ T cell populations were assessed in both the spleen and the tumor after re-challenge. In mice treated with [²²⁵Ac]-Compound D and checkpoint inhibitors, both the spleen and the tumor exhibited the presence of CD8+ T-cells. Importantly, an increase in the CD8+ T-cell frequency in the tumor, relative to controls, was observed. These results suggest that these combination treatments lead to improved levels of therapeutically effective CD8+ T cells.

Antigen-specific T-cells were detected and enumerated using an MHC class I tetramer assay. In this assay, MHC I molecules presenting an epitope specific to CT26 cells are labelled with biotin. In the presence of streptavidin, these MHC I molecules tetramerize. CD8+ T cells specific for the CD26 epitope are thereby labelled when their T-cell receptors bind to MHC I/CT26 epitope complexes within tetramers. Based on tetramer analysis, approximately 35%, 62%, and 75% of the CD8+ T cells were antigen-specific in mice treated with [²²⁵Ac]-Compound D/α-CTLA-4, [²²⁵Ac]-Compound D/α-PD-1, and [²²⁵Ac]-Compound D/α-CTLA-4/α-PD-1, respectively.

### Example 9. Synergy Between [²²⁵Ac]-Compound D1 and α-CTLA-4/PD-1 Treatment in the CT26 Syngeneic Mouse Model

TAB-199 was conjugated with Compound A2 (a bifunctional chelate represented by the structure shown below) and radiolabeled with [²²⁵Ac] using standard techniques known in the field to form [²²⁵Ac]-Compound D1.

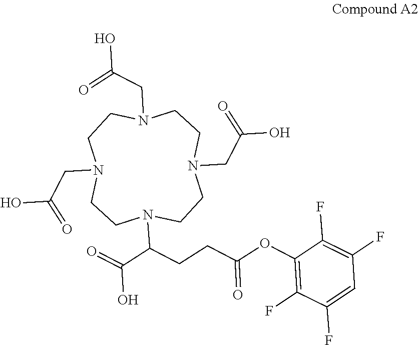 [SMILES: O=C(O)CN1CCN(CC(=O)O)CCN(C(CCC(=O)Oc2c(F)c(F)cc(F)c2F)C(=O)O)CCN(CC(=O)O)CC1]

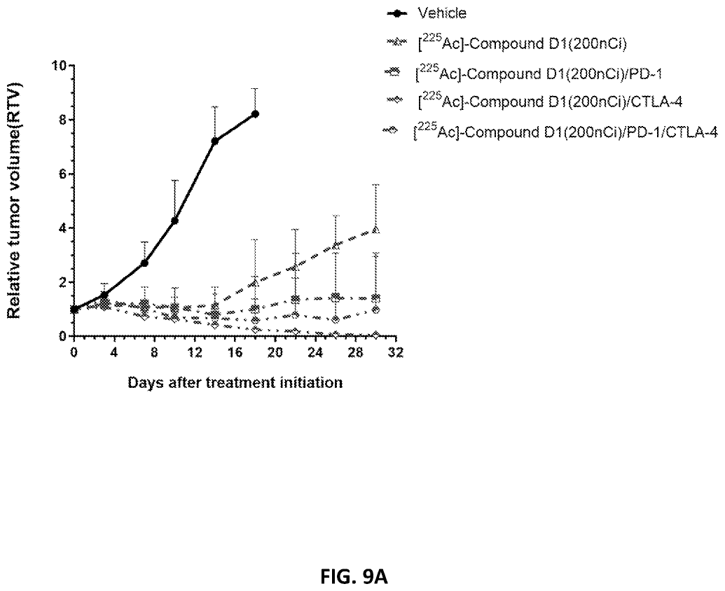

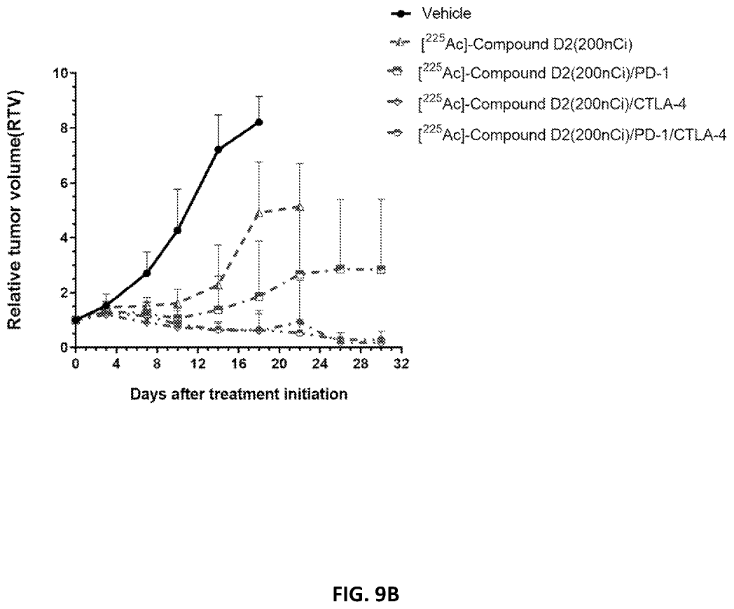

An in vivo synergy study was conducted to test the effect of [²²⁵Ac]-Compound D1 and checkpoint inhibitors, α-CTLA-4 and α-PD-1 antibodies, on relative tumor volume in the CT26 mouse model. Mice treated with [²²⁵Ac]-Compound D1 at the dose of 370 kBq/kg (or 200 nCi) demonstrated only transient tumor regression, followed by tumor regrowth. However, when [225Ac]-Compound D1 was co-administered at 370 kBq/kg with ether CTLA-4 or PD-1 or both, a synergistic effect was observed to show durable tumor regression-co-administration resulted in significantly smaller tumor volume when compared to treatment with [²²⁵Ac]-Compound D1. See FIG. 9A.

Of note, the dosage of 200 nCi in mice corresponds to 370 kBq/kg, which is equivalent to about 28 MBq in human (human equivalent dose).

### Example 10. Synergy Between [²²⁵Ac]-Compound D2 and α-CTLA-4/PD-1 Treatment in the CT26 Syngeneic Mouse Model

TAB-199 was conjugated with Compound A3 (a bifunctional chelate represented by the structure shown below) and radiolabeled with [²²⁵Ac] using standard techniques known in the field to form [²²⁵Ac]-Compound D2.

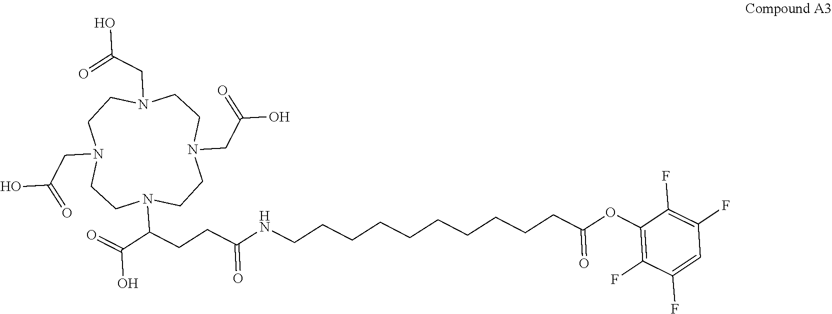 [SMILES: O=C(O)CN1CCN(CC(=O)O)CCN(C(CCC(=O)NCCCCCCCCCCC(=O)Oc2c(F)c(F)cc(F)c2F)C(=O)O)CCN(CC(=O)O)CC1]

An in vivo synergy study was conducted to test the effect of [²²⁵Ac]-Compound D2 and checkpoint inhibitors, α-CTLA-4 and α-PD-1 antibodies, on relative tumor volume in the CT26 mouse model. Mice treated with [²²⁵Ac]-Compound D2 at the dose of 370 kBq/kg (or 200 nCi) demonstrated only transient tumor regression, followed by tumor regrowth. However, when [225Ac]-Compound D was co-administered at 370 kBq/kg with ether CTLA-4 or a combination of PD-1 and CTLA-4, a synergistic effect was observed to show durable tumor regression—co-administration resulted in significantly smaller tumor volume when compared to treatment with [²²⁵Ac]-Compound D2. See FIG. 9B.

Of note, the dosage of 200 nCi in mice corresponds to 370 kBq/kg, which is equivalent to about 28 MBq in human (human equivalent dose).

### Example 11. Effects of Combination Therapy with [²²⁵Ac]-Labeled Conjugates Comprising AVE1642

[²²⁵Ac]-labeled radioimmunoconjugates comprising AVE1642 (a humanized monoclonal IGF-1R antibody), which can be prepared by conjugating AVE1642 with a bifunctional chelate such as Compound A1, Compound A2, or Compound A3 and then radiolabeling with [²²⁵Ac], can be tested in combination with checkpoint inhibitors (e.g., CTLA-4 antibodies and/or PD-1 antibodies) using protocols similar to those described in Examples 4-9. For example, effects on tumor volume, animal survival, cytokine expression, T-cell immunity (e.g., presence, amount, and/or function of tumor-associated antigen-specific CD8+ T cells), and protection against tumor re-challenge can be compared between combination therapy groups and monotherapy treatment and/or control groups.

### Example 12. Effects of Combination Therapy with [²²⁵Ac]-Labeled Conjugates Comprising FGFR3-Targeting Moieties

[²²⁵Ac]-labeled conjugates comprising an FGFR3-targeting moiety (e.g., an FGFR3 antibody or fragment thereof or a small molecule), which can be prepared by conjugating an FGFR3-targeting moiety with a bifunctional chelate such as Compound A1, Compound A2, or Compound A3 and then radiolabeling with [²²⁵Ac], can be tested in combination with checkpoint inhibitors (e.g., CTLA-4 antibodies and/or PD-1 antibodies) using experiments similar to those described in Examples 4-9, using a mouse model for an FGFR3-altered cancer. For example, effects on tumor volume, animal survival, cytokine expression, T-cell immunity (e.g., presence, amount, and/or function of tumor-associated antigen-specific CD8+ T cells), and protection against tumor re-challenge can be compared between combination therapy groups and monotherapy treatment and/or control groups.

### EQUIVALENTS/OTHER EMBODIMENTS

Those skilled in the art will recognize or be able to ascertain using no more than routine experimentation, many equivalents to the specific embodiments described herein. Such equivalents are intended to be encompassed by the following claims.

What is claimed is:

1. A method of treating a patient having cancer, said method comprising:
(i) administering to the patient an [²²⁵Ac]-radioimmunoconjugate, wherein the patient has received or is receiving one or more checkpoint inhibitors;
(ii) administering to the patient one or more checkpoint inhibitors, wherein the patient has received or is receiving an [²²⁵Ac]-radioimmunoconjugate; or
(iii) administering to the patient an [²²⁵Ac]-radioimmunoconjugate in combination with one or more checkpoint inhibitors,
wherein the [²²⁵Ac]-radioimmunoconjugate comprises ²²⁵Ac chelated with a compound having the formula: A-L¹-X-L²-Z—B, wherein:
A is a chelating moiety selected from the group consisting of DOTA (1,4,7,10-tetraazacyclododecane-1,4,7,10-tetraacetic acid), DOTMA (1R,4R,7R,10R)-α, α′, α″, α″′-tetramethyl-1,4,7,10-tetraazacyclododecane-1,4,7,10-tetraacetic acid, DOTAAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (10-(2-hydroxypropyl)-1,4,7-tetraazacyclododecane-1,4,7-triacetic acid);
L¹ is a bond or optionally substituted C₁₋₆ alkyl or C₁₋₆ heteroalkyl;
X is —C(O)NR¹—*, —NR¹C(O)—*, —OC(O)NR¹—*, —NR¹C(O)O—*, —NR¹C(O)NR¹—, —CH₂-Ph-C(O)NR¹—*, —NR¹C(O)-Ph-CH₂—*, —O—, or —NR¹—, wherein “*” indicates the attachment point to L², and each R¹ is independently hydrogen or C₁₋₆ alkyl;
L² is optionally substituted C₁₋₅₀ alkyl or C₁₋₅₀ heteroalkyl;
Z is —C(O)—, —CH₂—, —OC(O)—#, —C(O)O—#, —NR²C(O)—#, —C(O)NR²—#, or —NR²—, wherein “#” indicates the attachment point to B, and each R² is independently hydrogen or C₁₋₆ alkyl; and
B is a targeting moiety,
and wherein the [²²⁵Ac]-radioimmunoconjugate is administered at a dose of 10 kBq to 400 kBq/kg of body weight of said patient or is administered as a unitary dosage of 1-30 MBq to said patient.

2. The method of claim 1, comprising administering to the patient an [²²⁵Ac]-radioimmunoconjugate, wherein the patient has received or is receiving one or more checkpoint inhibitors.

3. The method of claim 1, comprising administering to the patient an [²²⁵Ac]-radioimmunoconjugate in combination with one or more checkpoint inhibitors.

4. The method of any one of claims 1-3, wherein the chelating moiety is DOTA.

5. The method of any one of claims 1-4, wherein the compound is represented by formula I:

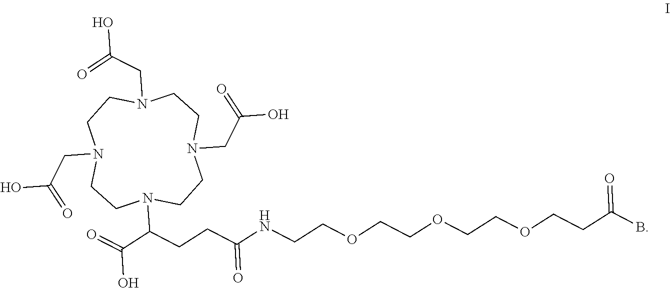

6. The method of any one of claims 1-4, wherein the compound is represented by formula II:

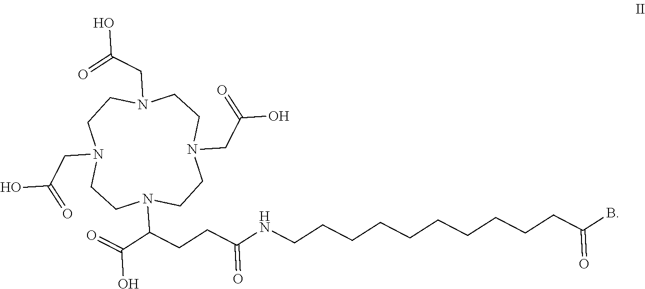

7. The method of any one of claims 1-6, wherein the targeting moiety comprises an antibody or antigen-binding fragment thereof.

8. The method of claim 7, wherein B is an insulin-like growth factor 1 receptor (IGF-1R) antibody or antigen-binding fragment thereof, an endosialin (TEM-1) antibody or antigen-binding fragment thereof, or a fibroblast growth factor receptor 3 (FGFR3) antibody or antigen-binding fragment thereof.

9. The method of claim 8, wherein B is an IGF-1R antibody or antigen-binding fragment thereof selected from the group consisting of figitumumab, cixutumumab, TAB-199, AVE1642, BIIB002, robatumumab, and teprotumumab, and antigen-binding fragments thereof.

10. The method of claim 9, wherein B is AVE1642 or an antigen-binding fragment thereof.

11. The method of any one of claims 1-10, wherein the [²²⁵Ac]-radioimmunoconjugate is administered at a dose of about 10 kBq to about 200 kBq/kg of body weight of said patient.

12. The method of any one of claims 1-10, wherein the [²²⁵Ac]-radioimmunoconjugate is administered at a dose of about 30 kBq to about 120 kBq/kg of body weight of said patient.

13. The method of any one of claims 1-12, wherein the one or more checkpoint inhibitors comprise a PD-1 inhibitor, a CTLA-4 inhibitor, or a combination thereof.

14. The method of claim 13, wherein the one or more checkpoint inhibitors comprise both a PD-1 inhibitor and a CTLA-4 inhibitor.

15. The method of claim 13 or 14, wherein the PD-1 inhibitor or the CTLA-4 inhibitor is an antibody.

16. The method of any one of claims 1-15, wherein the one or more checkpoint inhibitors is administered in a lower effective dose.

17. The method of any one of claims 1-16, wherein the [²²⁵Ac]-radioimmunoconjugate is administered in a lower effective dose.

18. The method of any one of claims 1-17, wherein the one or more checkpoint inhibitors comprise a PD-1 inhibitor administered at a dose of about 5 mg/kg to about 15 mg/kg.

19. The method of any one of claims 13-18, wherein the PD-1 inhibitor is pembrolizumab.

20. The method of any one of claims 1-19, wherein the one or more checkpoint inhibitors comprise both a PD-1 inhibitor and a CTLA-4 inhibitor, each administered at a dose of about 5 mg/kg to about 15 mg/kg.

21. The method of claim 9, wherein B is AVE1642 or an antigen-binding fragment thereof, and the one or more checkpoint inhibitors comprise a PD-1 inhibitor that is pembrolizumab.

22. The method of claim 21, wherein the [²²⁵Ac]-radioimmunoconjugate is administered at a dose of about 30 kBq to about 120 kBq/kg of body weight of said patient, and the PD-1 inhibitor administered at a dose of about 5 mg/kg to about 15 mg/kg.

23. The method of any one of claims 1-22, wherein the patient has a cancer selected from the group consisting of breast cancer, non-small cell lung cancer, small cell lung cancer, pancreatic cancer, head and neck cancer, prostate cancer, colorectal cancer, cervical cancer, endometrial cancer, sarcoma, adrenocortical carcinoma, neuroendocrine cancer, Ewing's Sarcoma, multiple myeloma, and acute myeloid leukemia.

24. The method of any one of claims 1-23, wherein the patient has a solid tumor expressing IGF-1R.

25. The method of any one of claims 1-24, wherein B is capable of binding to a tumor-associated antigen and said administering results in an increase in CD8+ T cells specific for the tumor-associated antigen.

26. The method of claim 25, wherein said administering results in at least 60% of the total CD8+ T cell population in a sample from the patient being specific for the tumor-associated antigen.

27. The method of claim 26, wherein the sample is a tumor sample.


SEQUENCE LISTING (24 sequences)

SEQ ID NO: 1 - protein, 16 aa, Artificial Sequence
  CDR-L1 of AVE1642
RSSQSIVHSNVNTYLE

SEQ ID NO: 2 - protein, 7 aa, Artificial Sequence
  CDR-L2 of AVE1642
KVSNRFS

SEQ ID NO: 3 - protein, 9 aa, Artificial Sequence
  CDR-L3 of AVE1642
FQGSHVPPT

SEQ ID NO: 4 - protein, 113 aa, Artificial Sequence
  Light chain variable region of AVE1642
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 5 - protein, 5 aa, Artificial Sequence
  CDR-H1 of AVE1642
SYWMH

SEQ ID NO: 6 - protein, 17 aa, Artificial Sequence
  CDR-H2 of AVE1642
EINPSNGRTNYNQKFQG

SEQ ID NO: 7 - protein, 15 aa, Artificial Sequence
  CDR-H3 of AVE1642
GRPDYYGSSKWYFDV

SEQ ID NO: 8 - protein, 124 aa, Artificial Sequence
  Heavy chain variable region of AVE1642
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 9 - protein, 10 aa, Artificial Sequence
  CDR-H1 of FGFR3 antibody
GFTFTSTGIS

SEQ ID NO: 10 - protein, 16 aa, Artificial Sequence
  CDR-H2 of FGFR3 antibody
GRIYPTSGSTNYADSV

SEQ ID NO: 11 - protein, 18 aa, Artificial Sequence
  CDR-H3 of FGFR3 antibody
TYGIYDLYVDYTEYVMDY

SEQ ID NO: 12 - protein, 20 aa, Artificial Sequence
  CDR-H3 of FGFR3 antibody
ARTYGIYDLYVDYTEYVMDY

SEQ ID NO: 13 - protein, 11 aa, Artificial Sequence
  CDR-L1 of FGFR3 antibody
RASQDVDTSLA

SEQ ID NO: 14 - protein, 7 aa, Artificial Sequence
  CDR-L2 of FGFR3 antibody
SASFLYS

SEQ ID NO: 15 - protein, 9 aa, Artificial Sequence
  CDR-L3 of FGFR3 antibody
QQSTGHPQT

SEQ ID NO: 16 - protein, 123 aa, Artificial Sequence
  Heavy chain variable region of FGFR3 antibody
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLV

SEQ ID NO: 17 - protein, 107 aa, Artificial Sequence [sequence omitted]

SEQ ID NO: 18 - protein, 125 aa, Artificial Sequence
  Heavy chain variable region of FGFR3 antibody
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARARTYGIYDLYVDYTEYVMDYWGQGTLV

SEQ ID NO: 19 - protein, 107 aa, Artificial Sequence
  Light chain variable region of FGFR3 antibody
DIQMTQSPSSLSASVGDRVTITCRASQDVDTSLAWYQQKPGKAPKLLIYSASFLYSGVPSRFSGSGSGTDFTLTISSLQPEDFATYYCQQSTGHPQTFGQGTKVEIK

SEQ ID NO: 20 - protein, 330 aa, Artificial Sequence
  Antibodoy heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 107 aa, Artificial Sequence [sequence omitted]

SEQ ID NO: 22 - protein, 150 aa, Artificial Sequence
  Light chain fragment of AVE1642
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKRTVAAPSVFIFPPSDEQLKSGTASVVCLLNNFYPREAK

SEQ ID NO: 23 - protein, 150 aa, Artificial Sequence
  Heavy chain fragment of AVE1642
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALG

SEQ ID NO: 24 - protein, 334 aa, Artificial Sequence
  Heavy chain fragment of FGFR3 antibody
TVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK